(19) United States
(12) Patent Application Publication
(10) Pub. No.: US 2004/0022861 A1
(43) Pub. Date: Feb. 5, 2004

(54) Process for production of nanoparticles and microparticles by spray freezing into liquid

(76) Inventors: Robert O. Williams III, Austin, TX (US); Keith P. Johnston, Austin, TX (US); Timothy J. Young, Midland, MI (US); True L. Rogers, Midland, MI (US); Melisa K. Barron, San Francisco, CA (US); Zhongshui Yu, Austin, TX (US); Jiahui Hu, Austin, TX (US)

Correspondence Address: Tracey B. Davies, VINSON & ELKINS LLP, 2300 First City Tower, 1001 Fannin, Houston, TX, 77002-6760, US

(21) Appl. No.: 10/273,730

(22) Filed: Oct. 18, 2002

Related U.S. Application Data
(63) Continuation-in-part of application No. PCT/US02/02894, filed on Jan. 30, 2002.
(60) Provisional application No. 60/345,473, filed on Oct. 19, 2001.
(60) Provisional application No. 60/264,988, filed on Jan. 30, 2001.

(51) Int. Cl. A61K 38/28; A61K 38/00; A61K 38/22

(52) U.S. Cl. 424/489; 514/3; 514/12

(57) ABSTRACT

The present invention provides a system and a method for the production of microparticles and nanoparticles of materials that can be dissolved. The system and method of the present invention provide quicker freezing times, which in turn produces a more uniform distribution of particle sizes, smaller particles, particles with increased porosity and a more intimate mixing of the particle components. The system and method of the present invention also produce particles with greater surface area than conventional methods. One form of the present invention provides a method for the preparation of particles. An effective ingredient is mixed with water, one or more solvents, or a combination thereof, and the resulting mixture is sprayed through an insulating nozzle located at or below the level of a cryogenic liquid. The spray generates frozen particles.

This application claims the benefit of U.S. provisional applications, serial Nos. 60/345,473 filed on Oct. 19, 2001, and 06/264,988 filed on Jan. 30, 2001, and is a continuation-in-part of PCT application PCT/US02/02894, each of which is specifically incorporated herein, in their entirety.

### BACKGROUND OF THE INVENTION

1. Field of the Invention

The present invention relates to drug particles and methods for their preparation. More particularly, the present invention relates to the preparation of drug particles utilizing spray freezing into liquid.

2. Description of Related Art

Small particle engineering enables an active pharmaceutical ingredient (API) to be incorporated into a formulation for targeted drug delivery. Powder micronization can also be used to increase the dissolution rates of poorly water-soluble drugs. Micronization procedures can modify particle size, porosity and density, and the API can be mixed with Pharmaceutical excipients using small particle technologies to maximize delivery to the desired target administration.

Particle formation technologies can be classified as either mechanical micronization processes or solution-based phase separation processes. Mechanical micronization methods include milling techniques such as that cited in U.S. Pat. No. 5,145,684 to Liversidge et al. However, friction generated during these milling processes can lead to either thermal or mechanical degradation of the API. Spray drying, another common method used to micronize drug substances, requires extremely high temperatures, on the order of 150° C., to remove the solvent from the drug following atomization. The elevated temperatures can accelerate degradation of the active ingredient.

Solution-based techniques require less particle handling and are often easier to scale up than conventional milling techniques. Reduced particle handling results in higher yields and simplifies cleaning and sterilization procedures. Furthermore, solution-based processes can be continuous or semi-continuous unlike milling, which is typically a batch process.

Solution-based particle formation techniques involve the use of conventional liquids, or compressed gases, near-critical liquids or supercritical fluids functioning either as solvents, antisolvents, or cryogenic media for ultra-rapid freezing. These techniques involve phase separation of solvent and API either by evaporation, rapid expansion, change in solvent composition or solidification by freezing. The spray configuration in many of these processes produces atomized droplets with relatively high surface areas compared to other methods. Thus phase separation and rapid nucleation tend to result in small primary particles or highly porous microparticles.

Several solution-based phase separation techniques utilizing compressed fluids have been developed. Such micronization techniques typically employ liquid or supercritical fluid carbon dioxide as solvents or antisolvent, and involve atomization of a solution into the carbon dioxide from the vapor space above the carbon dioxide. The active ingredient is either contained in the solution or in the carbon dioxide itself. Precipitation of the active ingredient results in amorphous or crystalline powders. Such precipitation techniques are commonly referred to as, for example, precipitation with a compressed antisolvent (PCA), precipitation by rapid expansion from supercritical solutions (RESS), rapid expansion from supercritical to aqueous solution (RESAS) and are described in S. Palakodaty and P. York, Phase behavioral effects on particle formation processes using supercritical fluids, Pharm. Res., v. 16, 976-85 (1999); D. J. Dixon, K. P. Johnston and R. A. Bodmeier, Polymeric materials formed by precipitation with a compressed fluid antisolvent, AIChE J., v. 39, 127-39 (1993); R. Bodmeier, J. Wang, D. J. Dixon, S. Mawson and K. P. Johnston, Polymeric microspheres prepared by spraying into compressed carbon dioxide, Pharm. Res., v. 12, 1211-17 (1995); B. Subramaniam, R. A. Rajewski and K. Snavely, Pharmaceutical processing with supercritical carbon dioxide, J. Pharm. Sci., v. 86, 885-90 (1997); and T. J. Young, S. Mawson, K. P. Johnston, I. B. Henriksen, G. W. Pace and A. K. Mishra, Rapid Expansion from supercritical to aqueous solution to produce submicron suspensions of water-insoluble drugs, Biotechnol. Prog., v. 16, 402-07 (2000).

The success of the above-identified techniques depends heavily, however, on the efficiency of atomization of the solution into the carbon dioxide. A disadvantage of such techniques when used with proteins and peptides, in particular, is that many organic solvents used to dissolve the API also denature proteins and peptides. Therefore, such techniques cannot lead to biologically active micronized protein or peptide powders. Even modified processes, utilizing an aqueous solution of proteins or peptides include the challenge of the low solubility of water in CO2, the need for large quantities of organic solvent, optimization of the mixing of multiple streams, and denaturation of the protein that can occur due to the exposure of the protein to the acidic CO2 (pH 3).

Another problem with the above-identified techniques is that, regardless of the API, they often require elevated temperatures to produce homogeneous precipitates, which can enhance degradation of thermally labile drugs. Another disadvantage is the low solubility of most organic solids in supercritical CO2, since low drug loading into the supercritical CO2 results in low production rates of powders.

To dry the particles resulting from the above-identified solution methods, techniques include spray-freeze drying processes described in U.S. Pat. No. 3,721,725 to Briggs et al. and No. 5,208,998 to Oyler, and M. Mumenthaler and H. Leuenberger, Atmospheric spray-freeze drying: a suitable alternative in freeze-drying technology, Int. J. Pharm., v. 72, 97-110 (1991). A disadvantage of spray freeze-drying processes is that peptides and proteins can be easily degraded or denature during freezing likely due to the phase separation of water and its soluble components followed by ice crystal growth. Similarly, the other heat-labile APIs are also negatively affected. The proteins may also be denatured or may aggregate upon exposure to an aqueous-air interface.

Current methods of preparing some API particles, including, for example, some peptide and protein particles, utilize such techniques as spray-drying and slow freezing followed by lyophilization. The alternative of spray-drying without freezing is deficient because it utilizes heat to evaporate the solvent system to produce the solid microparticles, and heat is known to degrade and denature peptides and proteins, as well as other heat-labile active ingredients. Slow freeze-induced phase separation also disadvantageously results in concentration of protein with the other excipients present in the aqueous solution. This causes, for example, high salt concentrations, pH swings caused by concentration of the buffer and other problems. Freeze concentration for slowly frozen protein solutions can be as much as 50 times the starting concentration. Even modified spray freeze drying processes utilize expensive halocarbon refrigerants and result in unsuitably large particles.

Another challenge to forming particles amenable to drug formulation is presented by poorly water-soluble compounds which are common among new chemical entities being investigated for therapeutic activity as APIs. Oil/water emulsions are frequently used in the pharmaceutical industry to enhance the overall concentration of poorly water-soluble and insoluble drugs, due to the high solubility of the API in the dispersed oil phase. Emulsion stability is a concern, however, and over time, emulsions often coalesce and settle. Additionally, the large volume of the oil and aqueous phases limits the overall drug concentration, potency and yield. To overcome these disadvantages, solvents are often removed from emulsion formulations by lyophilization. Further, freezing emulsions results in phase separation and destabilization of the APIs, and dry emulsions do not produce the same degree of dissolution enhancement as was achieved prior to lypohilization.

Accordingly, it would be an advantage in the art of particle engineering for the pharmaceutical industry to provide a process which results in the formation of stable small API-containing particles without the problems associated with the above-identified prior art.

All references cited in this application are specifically incorporated herein, in their entirety.

### SUMMARY OF THE INVENTION

In one aspect, the present invention is a system for preparing particles comprising a solution source comprising an effective ingredient; a vessel for holding a cryogenic liquid; and an insulating nozzle having an end and a tip, wherein the end of the insulating nozzle is connected to the solution source and the tip is placed at or below the level of the cryogenic liquid.

In a second aspect, the present invention is a method for spray freezing comprising mixing an effective ingredient with a solution agent, spraying the effective ingredient-solution agent mixture through an insulating nozzle located at or below the level of a cryogenic liquid, wherein the spray generates frozen particles. The particles can then be collected. The solvent is removed using, for example, lyophilization, to yield solid particles.

In yet a third aspect, the present invention is a particle that contains an effective ingredient and that has a size range of 10 nm to 10 μm, surface area of 0.5 m2/g to 115 m2/g as measured by the BET method, and a contact angle against purified water of 5 to 70 degrees. For embodiments including a peptide or protein as the API, the present invention also provides peptide or protein-containing particles with minimal monomer loss, in the range of as low as 0.1% or even lower, and minimal α-helix loss, as low as 0.1% or even lower.

In one aspect, the present invention provides a system and method for the production of microparticles and nanoparticles of materials. The present invention can provide quicker freezing times than those techniques described in the above-identified prior art, and can also produce a more uniform distribution of particle sizes, smaller particles, particles with increased porosity and a more intimate mixing of the particle components particles with higher surface area, and particles with a higher bioavailability. In addition, one embodiment of the present invention produces particles having enhanced stability.

In one aspect, the present invention provides a new process for producing microparticles and nanoparticles of pharmaceuticals by spray freezing into liquid (SFL). The SFL technique can be used to produce small particles containing one or more chemical agent(s) with or without an excipient. An excipient can be chosen to modify the intended function of the agent, such as to provide improved flow, enhance bioavailability or control/delay release of, for example, a pharmaceutical drug substance. Using the present SFL invention, poorly soluble agents, for example, have been made more water-soluble by controlling the particle size, degree of crystallinity and mixing with excipients. Furthermore, the particles produced using SFL have a more uniform morphology that can be manipulated to enhance delivery and bioavailability. For example, the SFL method produces microparticles and nanoparticles that are useful for deep lung drug delivery due to the combination of particle density, drug bioavailability and the low degradation found using the SFL method disclosed herein. While the SFL methods and techniques enhance the bioactivity of poorly soluble drugs, SFL may be applied to enhance and control particles produced from water soluble agents as well. Particles produced by the methods described have primary particles in the range of 10 nm to 10 microns after drying.

In another embodiment, the disclosed spray freezing process for producing microparticles comprises preparing a first emulsion comprising an encapsulation agent in a first solvent and an active agent in a first dispersed phase. Preparation of a first emulsion can be followed by spray freezing of the first emulsion or the preparation of a second emulsion comprising said first emulsion dispersed in a second solvent. The second emulsion, which can optionally be a double emulsion, can subsequently be spray frozen to produce said microparticles. In preferred embodiments a first or second emulsion is spray frozen into a cryogenic liquid.

In certain embodiments the active agent being formulated into the nanoparticles or microparticles of the present invention can be any active pharmaceutical ingredient (API) including, but not limited to, peptides, nucleic acids, proteins, antibiotics, gene therapy agents, small molecule APIs, catalysts, adsorbents, pigments, coatings, personal care products, abrasives, particles for sensors, metals, alloys, ceramics membrane materials, nutritional substances, anti-cancer agents, fertilizers, pesticides, and herbicides.

Also contemplated by the present invention are any pharmaceutical compositions including the particles produced as disclosed herein.

### BRIEF DESCRIPTION OF THE FIGURES

FIG. 1 depicts a schematic diagram of an apparatus for spray freezing into liquid in accordance with the present invention;

FIG. 2 depicts an apparatus for removing the solvent from the particles produced in accordance with the present invention; and

FIG. 3 depicts an apparatus capable of continuous flow operation in accordance with the present invention.

FIG. 4 illustrates an exemplary method of encapsulating an active agent in a microparticle.

FIG. 5 is an exemplary size distribution diagram of lysozyme loaded microparticles produced by exemplary methods.

FIG. 6 illustrates an exemplary effect of insulin concentration (internal aqueous phase), polymer type and polymer concentration (oil phase) on the loading degree of insulin within microparticles.

FIG. 7 illustrates an exemplary effect of different types of polymer and polymer concentration within the organic phase on the loading degree of insulin.

FIG. 8 illustrates an exemplary effect of applied emulsification energy during the second emulsification step on the loading degree of insulin within the DL-PLGA microparticles.

FIG. 9 illustrates cumulative release of insulin from exemplary microparticles.

FIG. 10 illustrates an exemplary HPLC chromatogram of A) freshly prepared insulin solution as the control, B) insulin extracted from insulin loaded microparticles produced by SFL technology.

FIG. 11 illustrates the cumulative release of BSA from PLGA microparticles for example 13.

FIG. 12 illustrates the cumulative release of BSA from PLGA microparticles for example 14.

FIG. 13 illustrates the cumulative release of Insulin from PLGA microparticles for example 15.

FIG. 14 is a schematic illustration of the SFL process used to make the micronized SFL powders from emulsion: a) feed emulsion, b) liquid nitrogen, c) 127 μm I.D.×15-cm long insulated PEEK nozzle, d) syringe pump, e) high pressure valve and f) atomized frozen microdroplets.

FIG. 15 shows the XRD patterns from emulsions. 15a) shows XRD patterns of micronized SFL powders from emulsion: bulk danazol (X), the co-ground physical mixture (P), micronized SFL A powder (A), micronized SFL B powder (B), micronized SFL C powder (C), micronized SFL D powder (D) and micronized SFL E powder (E). 15b) shows XRD patterns of slowly frozen agglomerates from emulsion: bulk danazol (X), co-ground physical mixture (P), slowly frozen A agglomerate (A), slowly frozen B agglomerate (B) and slowly frozen C agglomerate (C). 15c) shows XRD patterns of micronized SFL powder and controls from solution: bulk danazol (X), co-ground physical mixture (P), micronized SFL powder from solution (A) and slowly frozen control from solution (B).

FIG. 16 shows SEM micrographs of a) bulk danazol, b) co-ground physical mixture, c) micronized SFL A powder, d) micronized SFL D powder, e) slowly frozen A agglomerate, f) micronized SFL powder from solution g) slowly frozen agglomerate from solution.

FIG. 17 shows TEM micrographs of a) the micronized SFL D powder and b) the micronized SFL powder from solution.

FIG. 18a) shows dissolution profiles of micronized SFL powders from emulsion: bulk danazol (X), co-ground physical mixture (⋄), micronized SFL A powder (▪), micronized SFL B powder (♦), micronized SFL C powder (▴), micronized SFL D powder (●) and micronized SFL E powder (+). b) Dissolution profiles of slowly frozen agglomerates from emulsion: bulk danazol (X), co-ground physical mixture (⋄), slowly frozen A (▪), slowly frozen B (♦) and slowly frozen C (▴). c) Dissolution profiles of micronized SFL powder and controls from solution: bulk danazol (X), co-ground physical mixture (⋄), micronized SFL powder from solution (◯), slowly frozen agglomerate from solution (□).

### DETAILED DESCRIPTION OF THE INVENTION

While the making and using of various embodiments of the present invention are discussed here in terms of an apparatus, method for producing microparticles and nanoparticles, and particles of any API including proteins and peptides having enhanced stability, it should be appreciated that the present invention provides many inventive concepts that can be embodied in a wide variety of specific contexts. The specific embodiments discussed herein are merely illustrative of specific ways to make and use the invention and are not meant to limit it in any way.

For the purposes of this application, the term “water soluble” is defined as requiring less than 1000 part water to dissolve 1 part solute. The term “poorly water soluble” is defined as requiring 1000 or more parts water to dissolve 1 part solute. The term “solution” as used in this application is meant to include suspensions and emulsions, including multiple-emulsions, as well as solutions.

The present invention provides a system and a method for producing extremely small particles, that is, microparticles and nanoparticles, by spray freezing a solution or suspension at the surface of or within a cryogenic liquid. The solution contains an effective ingredient and a solution agent, and can also contain any of a variety of adjuvants and/or excipients.

Apparatus and Methods of Particle Manufacture

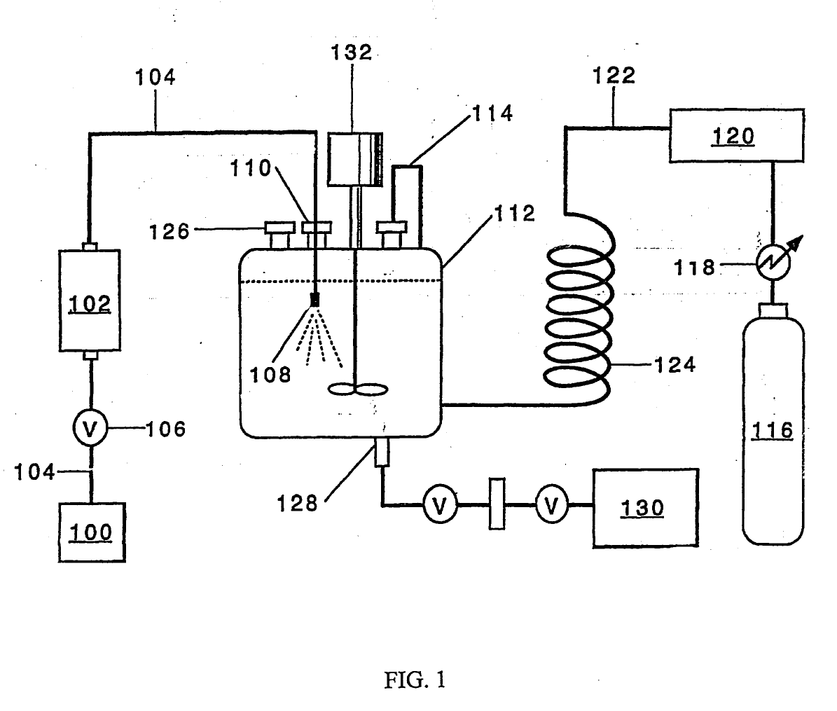

FIG. 1 diagrams an exemplary embodiment of an apparatus for use in the production of the particles of the present invention. The effective ingredients and any excipients are dissolved and placed in the solution source 100 that is connected to a pump 102 via a delivery line 104 and a valve 106. The pump 102 can be, for example, an HPLC pump, a syringe pump, or any other pump 102 capable of delivering the solution from the solution source 100 at the desired rate. The delivery line 104 can be stainless steel tubing or any other material suitable for use with a HPLC pump or a syringe pump.

The pump 102 is connected to an insulating nozzle 108 via delivery line 104. Located between solution source 100 and nozzle 108 can be a valve 110. For the purposes of this application, an “insulating nozzle” is any nozzle that does not become blocked when positioned at or within a cryogenic liquid while a solution is being passed through it, and that has no active defrosting apparatus connected to it. In one particular embodiment the insulating nozzle 108 tip is positioned substantially at the interface of the cryogenic liquid, thereby decreasing the amount of insulative property that the insulating nozzle 108 will require to prevent the solution that is traversing the insulating nozzle 108 from freezing. As will be apparent to those of skill in the art, in light of the present disclosure, the properties of the solution (pH, salt content, excipient, solubilizing agents, solvent) will affect the solutions ability to freeze. In addition to intrinsic factors or characteristics of the solution, the manner of application through the insulating nozzle 108 will affect freezing, for example, flow rate and pressure.

The solvent or solution agent used in the solution can be aqueous, such as water, one or more organic solvents, one or more emulsions, or a combination thereof. When used, the organic solvents can be water soluble or non-water soluble. Suitable organic solvents include but are not limited to ethanol, methanol, tetrahydrofuran, acetonitrile, acetone, tert-butyl alcohol, dimethyl sulfoxide, N,N-dimethyl formamide, diethyl ether, methylene chloride, ethyl acetate, isopropyl acetate, butyl acetate, propyl acetate, toluene, hexanes, heptane, pentane, and combinations thereof.

The insulating nozzle 108 has an end that is defined as the location where material to be spray frozen enters the insulating nozzle 108. The insulating nozzle 108, also has a tip, which is defined as the location where the material to be spray frozen exits the insulating nozzle 108. The insulating nozzle 108 is positioned such that the insulting nozzle tip is at or below the top surface of a cryogenic liquid in vessel 112.

The vessel 112 can be, for example, a sealed pressure vessel, as shown, for working with cryogenic liquids that liquefy at pressures above atmospheric, for example, carbon dioxide, or it can be an open top vessel for cryogenic liquids that are liquids at ambient pressures, for example, inorganic or organic molecules, such as nitrogen or ethanol, respectively. The vessel 112 will generally act as a static reservoir, but can permit continuous flow of the cryogenic liquid.

The insulating nozzle 108 can be composed of, for example, a molded tip. Molded tips for use with the invention can be made primarily of, for example, a polyetheretherketone such as PEEK. Another example is a molded tip made of polyether block amide such as PEBAX or a thermoplastic polyurethane elastomer such as PELLETHANE on tubing made of a polyetheretherketone. The insulating nozzle 108 can also be made of any nonreactive insulating material, for example, nylon, rayon, polyester, glass or the like, and can even be coated with TEFLON or another material with a substantially low coefficient of friction. It can also be made of a metal with or without outer insulation. The inner diameter of the nozzle can range from about one micron to about one centimeter. The thickness of the insulating nozzle 108 ranges from about 0.5 mm to about 10 cm. The insulating nozzle 108 is generally attached to the delivery line 104 coming from the solution source 100 by a sealed tubing having a connection capable of withstanding operating pressures. However, any attachment method that can withstand the pressure within the line is appropriate. In certain embodiments a nozzle orifice can be in the approximate range of 20 μm to approximately 2 mm.

Alternative embodiments include the use of a nozzle such as that used to spray an emulsion into a vapor phase above the meniscus between the vapor and liquid of a cryogenic medium, such nozzles are known to those of skill in the art. Alternative embodiments can use a sonicating nozzle or a two-fluid nozzle to spray an emulsion above the meniscus of a cryogenic medium or into a cryogenic medium. Examples of alternative atomization nozzles include, but are not limited to, external air (or gas) atomizers (for example, Glatt Model 014 available from Ortho Liquid System, NC, Models SUE iSA; SU2A and SU2 available from Spray Systems Co., Wheaton, Ill.), internal air atomizers (for example, SU12; Spray Systems Co.) and pressure atomization nozzles (for example, Type SSTC Whirl Jet Spray Drying Nozzles; Spray Systems Co., Wheaton, Ill.). Other atomization nozzles include, but are not limited to single fluid atomization assemblies, for example, ultrasonic atomization, can be utilized provided that conditions that yield equivalent atomization energy and performance are employed.

Several other ports can be included in the vessel 112. One such port can serve as an inlet port for the cryogenic gas or liquid 114. The cryogenic liquid 114 can be pumped into the vessel 112 from a compressed gas tank 116 that is connected to a regulator 118 and a filter and drying unit 120.

The cryogenic liquid 114 passes through tubing 122 that passes through a chilled ethanol dry ice bath 124. Other ports that can be connected to the vessel include a release valve 126 and a pressure and temperature transducer port 128 that is connected to display equipment 130. A stirrer 132 can also be used in this version of the present invention.

In operation, the effective ingredient or ingredients are dissolved, suspended or emulsified in a solvent or one or more solvents or cosolvent system (organic, aqueous—organic or aqueous system) and placed in a solution source, for example, solution source 100. The effective ingredient and solvent can be presterilized before particle formation. Likewise, the entire, sealed apparatus can be sterilized prior to use, thereby providing particles that can used directly or packaged in a sterile manner for future use. The solution source 100 can be made, for example, stainless steel and can contain one or more low- or high-pressure fittings.

The solution source 100 can itself act as a pump by adding a piston that drives the solution within the solution source into or toward the insulating nozzle 108. In one example, pressure within the solution source 100 can be increased by pressurized gas, for example, carbon dioxide. The delivery of the gas and the pressure in the solution source 100 can be controlled by, for example, a digital syringe, or even an HPLC pump. The choice of pump will be apparent to, and readily within, the skills of the artisan.

TABLE 1
Examples of SFL conditions for the creation of microparticles
and nanoparticles using the present invention.

| | | | | | Liquid | Cryogen |
|---|---|---|---|---|---|---|
| Agent | Excipient | Temperature | Pressure | Nozzle | Flow Rate | Flow Rate |
| Albuterol | 10 percent | −40 C. | 1000 psig | 0.005″ | 1-10 | 35 ml/min |
| sulfate, | PEG; | | | | ml/min | |
| Triamcinolone | 18,500 | | | | | |
| Acetonide, | MW | | | | | |
| Insulin | | | | | | |
| Albuterol | 10 percent | −40 C. | 1000 psig | 0.005″ | 1-10 | 17.5 |
| sulfate, | PEG; | | | | ml/min | ml/min |
| Triamcinolone | 18,500 | | | | | |
| Acetonide, | MW | | | | | |
| Insulin | | | | | | |
| Albuterol | 10 percent | −40 C. | 1000 psig | 0.005″ | 1-10 | 5 ml/min |
| sulfate, | PEG; | | | | ml/min | |
| Triamcinolone | 18,500 | | | | | |
| Acetonide, | MW | | | | | |
| Insulin | | | | | | |
| Albuterol | 10 percent | −40 C. | 1000 psig | 0.005″ | 1-10 | 2 ml/min |
| sulfate, | PEG; | | | | ml/min | |
| Triamcinolone | 8,000 MW | | | | | |
| Acetonide, | | | | | | |
| Insulin | | | | | | |
| Albuterol | 10 percent | −40 C. | 1000 psig | 0.005″ | 1-10 | 5 ml/min |
| sulfate, | PEG; | | | | ml/min | |
| Triamcinolone | 8,000 MW | | | | | |
| Acetonide, | | | | | | |
| Insulin | | | | | | |
| Albuterol | 2 percent | −40 C. | 1000 psig | 0.005″ | 1-10 | 2 ml/min |
| sulfate, | PEG; | | | | ml/min | |
| Triamcinolone | 18,500 | | | | | |
| Acetonide, | MW | | | | | |
| Insulin | | | | | | |
| Albuterol | 2 percent | −40 C. | 1000 psig | 0.0025″ | 1-10 | 2-35 |
| sulfate, | PEG; | | | | ml/min | ml/min |
| Triamcinolone | 18,500 | | | | | |
| Acetonide, | MW | | | | | |
| Insulin | | | | | | |
| Albuterol | 10 percent | −40 C. | 1000 psig | 0.005″ | 1-10 | 2-35 |
| sulfate, | PEG; | | | | ml/min | ml/min |
| Triamcinolone | 18,500 | | | | | |
| Acetonide, | MW | | | | | |
| Insulin | | | | | | |

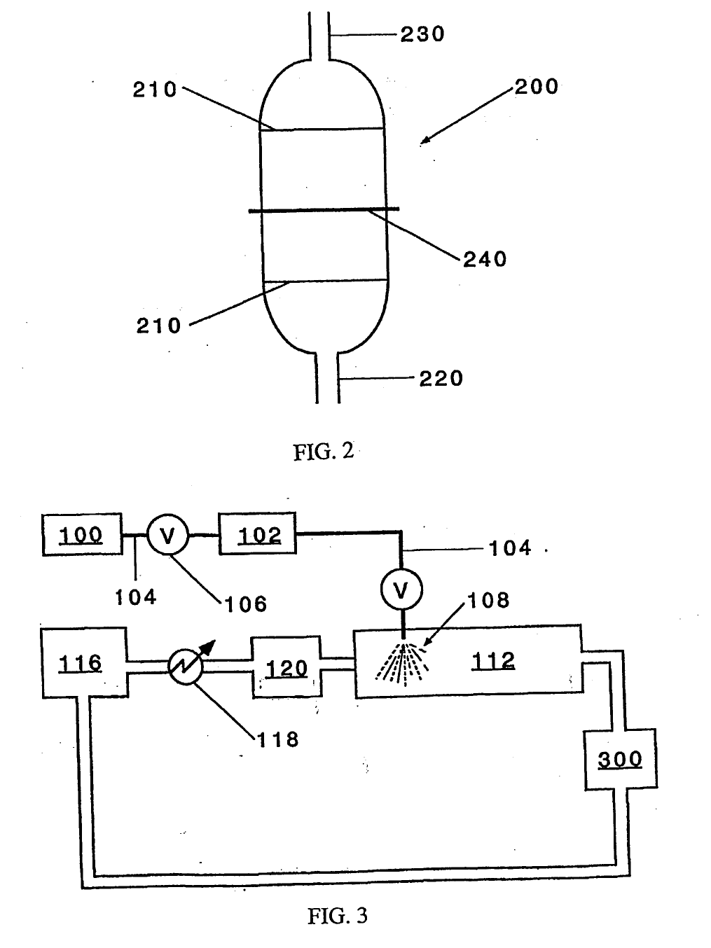

A filter drying unit 200 is depicted in FIG. 2. The freeze-sprayed particles are transferred to the unit 200 so that they reside between two porous filter elements 210. The porous filter elements 210 can be of any material that allows retention of the particles within the unit 200. Examples of a suitable porous filter element 210 include a glass fit, a blown-through filter cloth, a screen, a porous disc, or other suitable porous filter elements, as well as other components which can be envisaged by those of skill in the art in light of the present disclosure.

The frozen particles are introduced into the unit 200 via an access joint 240. The unit 200 is attached to a source of cooled gas via inlet 220. The incoming gas will most often be cooled to a temperature below the melting point of the particles so that greatly reduced redissolution takes place. If necessary, the entire unit 200 can be cooled externally, by immersion in a suitable refrigerant. The cooled gas passes through the unit 200 from the bottom to top and passes out of the unit 200 via outlet 230. In this manner the solvent is removed from the frozen particles.

The process can also be operated in a continuous flow mode as depicted in FIG. 3. By using a continuous flow of the solution source 100 and/or the cryogenic liquid the throughput of the compound that is being spray frozen can be increased dramatically with the incorporation of a filtration unit 300 for the removal of the frozen particles. As will be known to those of skill in the art of pharmaceutical automation, it will be useful that the system is self-contained and capable of being sterilized. In this manner the spray freezing occurs in continuous mode and the microparticles or nanoparticles are captured downstream from the spray freezing site. The capture mechanism can include a bucket or conveyor belt system that takes the particles and places them in storage for subsequent processing or for removal of the solvent.

Whether using the batch or the continuous flow method, the size, shape, density, porosity and flowability of the particles can be modified by, for example, varying the solvent, and the relative proportion of any cosolvent (when one is used). It is also possible to alter the morphology of the particles by varying the temperature of the solution in the solution source, the concentration of the solutes, the flow rate of the solution through the nozzle, the inner diameter and/or shape of the nozzle and the pressure drop between the transfer line and the nozzle, to name a few options. Those of skill in the art will recognize that known parameters can be used, with minimal investigation, to obtain desired particle sizes.

Embodiments of a process for manufacture of particles, such as microparticles, by spray freezing in vapor or spray freezing into a cryogenic media are exemplified and described herein. In one embodiment, a solution or dispersion of an active agent in an appropriate water based medium (internal aqueous phase) is emulsified with a solution of polymers in an organic solvent (organic phase) using a high shear homogenizer at high speed (approximately 8000-14000 rpm for 10-300 seconds). In certain embodiments, a high concentration of active agent is desired. A high concentration of active agent is useful in some embodiments, to minimize the internal aqueous phase volume needed to incorporate a larger amount of active agent into particles. The solubility of active agents in an internal aqueous phase can also be optimized using various methods such as pH adjustment. In alternative embodiments, an active agent can be soluble in a first organic solvent and that a polymer can be preferentially soluble in a second organic solvent. The first and second solvent can not be readily miscible in each other, thus forming an oil in oil emulsion.

Embodiments of the invention can use various types of additional agents, excipients, adjuvants, or combinations thereof, as discussed herein, to stabilize, protect, or generally enhance the activity or other characteristics of a microparticle. In alternative embodiments, emulsions and multiple emulsions of the invention can be spray frozen (SF) into a vapor phase above the meniscus of a cryogenic media or below the level of the cryogenic media. Described herein are examples of SF and SFL of emulsions, including double emulsions; solutions; dispersions; and/or solid suspensions. Preferred embodiments of the invention use SFL of emulsions to produce microparticles. In one embodiment, the invention uses SFL of double emulsions to produce microparticles.

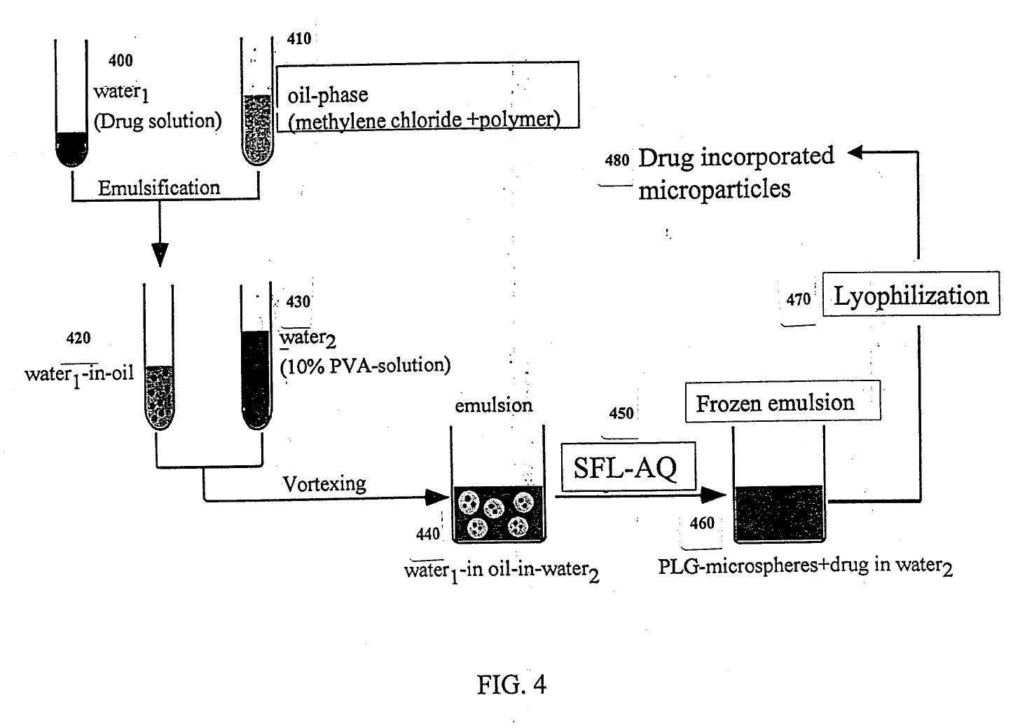

Embodied methods comprise a combination of emulsion techniques, particularly double emulsion techniques, and a cryogenic process for production of active agent loaded microparticles. FIG. 4 illustrates an exemplary embodiment of the methods of the invention. Briefly, an aqueous solution or dispersion of agent 400, for example protein/peptide, can be emulsified into an organic phase 410 comprising a biodegradable polymer dissolved in an organic solvent, for example methylene chloride. The resulting water/organic (‘w/o”) emulsion 420 is added to a solution of water and stabilizer 430, (for example, polyvinyl alcohol (PVA)), and then mixed to obtain the w/o/w double emulsion 440. The double emulsion 440 is subsequently frozen by spray freezing into a cryogenic liquid 450, for example liquid nitrogen. The resultant microparticle from SFL of a double emulsion 460 is then collected and lyophilized 470. During lyophilization water and organic solvent are removed. The resulting powder contains the active agent microencapsulated in a polymer 480. The dry product can be re-suspended in water and washed with distilled water and lyophilized over night to obtain a free flowing, water dispersible powder. The microparticles can be injected to provide a delivery system of water-soluble molecules, such as proteins/peptides. In addition the particles offer a controlled released system for hormone therapy, cancer therapy, vaccination, and/or other therapeutic methods, as well as agricultural agents, as described below.

In one embodiment a w/o emulsion can be added to a solution containing a stabilizer, for example polyvinyl alcohol (PVA), and mixed to obtain the w/o/w double emulsion. The presence of PVA or the like can stabilize a double emulsion. The concentration (1-6% w/w) and molecular weight of PVA can be optimized for particular applications. In certain embodiments the PVA concentration can be in the approximate range of 1% to 6%, preferably 2% to 4%, and more preferably 3% to 3.5%. As the concentration of PVA increases the solution becomes more viscous and more difficult to break up. The multiple emulsion formation will be subsequently sprayed and atomized into (beneath the surface) of a cryogenic liquid. The time interval between the formation of the double emulsion and atomization it into the liquid nitrogen is critical and can be in the approximate range of 30 seconds to approximately 35 minutes.

Atomization of a double emulsion into a cryogenic media, in some embodiments, results in an improvement of entrapment efficiency and loading degree due to rapid freezing which prevents the migration of water droplets containing active agent/excipient from an internal aqueous phase into an external aqueous phase. Further improvement of stability and retained bioactivity of active agent/excipient can result from very rapid freezing. The rapid freezing can limit the formation of ice crystals and instead lead to glassy non-crystalline material. Glassy materials produce less denaturation than do materials with ice crystals. As the solid aqueous phase separates from the liquid aqueous phase, the concentration of the dissolved materials in the liquid phase increases, which can lead to denaturation and/or degradation of an active agent. Thus, it is preferable to freeze the system rapidly. The spray atomization technique is effective for achieving rapid freezing. Sugars and other excipients can be added to the aqueous or organic phases to further limit denaturation.

Particles resulting from the spray freezing of emulsions, including multiple emulsions, can be collected and lyophilized. The lyophilizing cycle is chosen over an appropriate time period, usually longer than 24 hours, but time periods less than 24 hours can be appropriate in some circumstances. The dry product will be then re-suspended in water and washed two or three times with distilled water to remove excess materials, for example PVA and non encapsulated active agent or excipient. The particles can be collected by centrifugation and subsequently lyophilized overnight to obtain water dispersible powders.

Particles produced by methods described herein can vary in particle size depending on the parameters and materials used during production. Typically, particle sizes can include, but are not limited to an approximate range of 0.5 μm to 1000 μm, preferably 1 μm to 80 μm, with smaller particle sizes achievable under appropriate conditions. Particle size refers to a volume median particle size as determined by conventional particle size measuring techniques known to those skilled in the art, such as, laser diffraction, photon correlation spectroscopy, sedimentation field flow fractionation, disk centrifugation or electrical sensing zone. Laser diffraction is preferred. Microparticles typically, refer to particles having a particle size of less than 1000 μm.

In certain embodiments the concentration of a polymer(s) in the oil phase can be increased resulting in reduced solidification times. Also, increased viscosity in the oil phase caused by the increased concentration of polymer(s) will decrease the loss of active agent and contribute to enhanced encapsulation efficiency. The concentration of polymer needed to achieve particular encapsulation efficiency relative to a particular loading degree can be optimized for the appropriate active agent(s).

Double emulsions can include, but are not limited to water in oil in water (w/o/w) emulsion. A w/o/w emulsion can be produced by emulsification of an aqueous solution of an active agent into an organic solution of an encapsulation polymer to produce a first emulsion. The first emulsion can be subsequently added to an aqueous solution that can contain an excipient and/or adjuvant to produce a second emulsion (double emulsion). The second emulsion can be spray frozen using methods described herein and/or other methods known in the art. The particles can then be further processed by collecting and drying the particles. Preferably the particles are collected and lyophilized. The dried particles can be re-suspended and washed in an appropriate solvent, preferably water. The washed particles can be subjected to further lyophilization. Particle compositions prepared as described herein can be used as a delivery system for active agents, as well as providing for the controlled release of an active agent.

Water, aqueous buffers, organic solvents and mixtures thereof are suitable choices of solvents for use in the dispersed system. The choice of solvent can be determined for the particular biologically active agent being used and the type of dispersed system desired. A preferred solvent is a buffer, which can be either partially or completely removed. Buffers include, for example, ammonium salts, such as ammonium bicarbonate, and sodium salts, such as sodium bicarbonate.

Cryogenic Media

Any non-reactive cryogenic liquid is appropriate for use in the present invention. In preferred embodiments a cryogenic medium is a cryogenic liquid. Cryogenic liquids are defined as any material (organic or inorganic) that remains liquid below the freezing point of water. Non-reactive is defined for the purposes of this application as not undergoing a chemical reaction with any of the components of the solution that is to be spray frozen or spray frozen into a cryogenic medium. Non-limiting examples include carbon dioxide, nitrogen, ethane, isopentane, propane, helium, halocarbons, liquid ammonia and argon. A cryogenic liquid can be held statically in a vessel, or can be circulated through an appropriate vessel that is equipped with a filter to collect the particles that are formed.

Polymers

Certain embodiments of the invention use a polymer or a combination of polymers to produce microparticles. Microparticles of the invention can encapsulate an active agent in any polymer, co-polymer or polymer blend, preferably a biodegradable one, with high encapsulation efficiency, high loading, uniform active agent delivery, minimal burst effects, uniform porosity and combinations thereof. In other embodiments active agents can be incorporated as therapeutics in a microparticle delivery systems, such as sustained release depot systems and pulmonary delivery systems. In preferred embodiments, biodegradable polymers can be used to provide at least a microparticle that is biocompatible and degrades in vivo by forming nontoxic monomers and a release rate of entrapped active agents that can be controlled by varying the molecular weight of the polymer(s) and/or the copolymer ratios of the microparticles. Biodegradable, as defined herein, means the composition will degrade or erode in vivo to form smaller chemical species. Degradation can result, for example, by enzymatic, chemical and/or physical processes. Furthermore, administration of an active agent by injection is possible if it is dispersed into microparticles (Service, 1994; Aftabroushad and Doelker, 1994; and Morris and Steinhoff, 1994). It is contemplated that in other instances where biodegradation is not required other polymers known to the skilled artisan can be used.

The type and concentration of polymer can be important and can be optimized in order to improve the encapsulation efficiency and particle formation. A combination of two or more different polymers can be used to optimize particle formation. Alternatively, different molecular weights of the same type of polymer can be used in order to optimize particle formation. The type and molecular weight of polymers and encapsulation methodology largely influence the entrapment efficiency and release of encapsulated material from the polymeric microparticles. Particles prepared from more hydrophobic polymers or more crystalline polymers can degrade more slowly. In preferred embodiments a polymer is a biodegradable polymer. Generally, the biodegradation times of poly (lactide-co-glycolide) polymers are summarized in Table 2.

TABLE 2
Biodegradation Rate Of Exemplary Polymers

| | Approximate time |
|---|---|
| Polymer | for biodegradation (Month) |
| Poly (L-lactide) | 12-24 |
| Poly (glycolide) | 8-16 |
| Poly (lactide-co-glycolide) 85:15 | 5 |
| Poly (lactide-co-glycolide) 50:50 | 2 |

Acceptable molecular weights for polymers used in this invention can be determined by a person of ordinary skill in the art taking into consideration factors such as the desired polymer degradation rate, physical properties such as mechanical strength, arid rate of dissolution of polymer in a solvent. Typically, an acceptable range of molecular weights is, in the approximate range, of 2,000 Daltons to approximately 2,000,000 Daltons. In preferred embodiments, the polymer is a biodegradable polymer or copolymer. In a more preferred embodiment, the polymer is a poly(lactide-co-glycolide) (hereinafter “PLG”) with a lactide:glycolide ratio of about 1:1 and a molecular weight in the approximate, range of 3,000 Daltons to approximately 70,000 Daltons. In a yet more preferred embodiment, the molecular weight of the PLG used in the present invention has a molecular weight in the approximate range of 5,000 Daltons to approximately 42,000 Daltons.

Biocompatible, non-biodegradable polymers suitable for a sustained release device include non-biodegradable polymers selected from the group consisting of polyacrylates, polymers of ethylene-vinyl acetates and acyl substituted cellulose acetates, non-degradable polyurethanes, polystyrenes, polyvinyl chloride, polyvinyl fluoride, poly(vinyl imidazole), chlorosulphonate polyolefins, polyethylene oxide blends and copolymers thereof.

Exemplary polymers that can be used in the practice of the invention include biodegradable polymers such as poly fatty acid esters, for example, homopolymer of fatty acid (for example, polylactic acid, polyglycolic acid, polycitric acid, polymalic acid, or the like) or copolymers of two or more fatty acids (for example, copolymer of lactic acid/glycolic acid, copolymer of 2-hydroxybytyric acid/glycolic acid, or the like), a mixture of the homopolymer and/or copolymer (for example, mixture of poly lactic acid and copolymer of 2-hydroxybytyric acid/glycolic acids, or the like). Examples of the fatty acid include alpha-hydroxycarboxylic acids (for example, glycolic acid, lactic acid, 2-hydroxybutyric acid, 2-hydroxyvaleric acid, 2-hydroxy-3-methylbytyric acid, 2-hydroxycaproic acid, 2-hydroxyisocaproic acid, 2-hydroxycaprylic acid, or the like), cyclic dimers of alpha-hydroxycarboxylic acids (for example, glycolide, lactide, or the like), hydroxydicarboxylic acid (for example, malic acid, or the like), hydroxytricarboxylic acid (for example, citric acid; or the like) and so on, poly-alpha-cyanoacrylate, polyalkylene oxyalates (for example, polytrimethylene oxyalate, polyteteramethylene oxyalate, or the like), poly ortho esters, poly ortho carbonates and other polycarbonates (for example, polyethylene carbonate, polyethylenepropylene carbonate, or the like), polyamino, acids (for example, poly-γ-benzyl-L-glutamic acid, poly-L-alanine, poly-γ-methyl-L-glutamic acid, or the like) and the like. Further examples of the biocompatible polymers include polyacrylic acid, polymethacrylic acid, copolymer of acrylic acid and methacrylic acid, silicon polymer; dextranstearate, ethylcellulose, hydroxypropylmethylcellulose, acetylcellulose, maleic anhydride copolymers, ethylene-vinylacetate copolymers, polyvinyl acetate, polyvinyl alcohol, polyacrylamide and the like. These polymers can be used alone or in combination thereof. They can be used in the form of a copolymer or mere mixture of these two or more polymers. Among these polymers, in particular, poly fatty acid esters, poly-alpha-cyanoacrylate is preferred. Most preferred examples include poly fatty acid esters.

Among these poly fatty acid esters, in particular, homopolymers of alpha-hydroxycarboxylic acids, cyclic dimers of alpha-hydroxycarboxylic acids; copolymers of two or more alpha-hydroxycarboxylic acids, cyclic dimers of alpha-hydroxycarboxylic acids; and a mixture of the homopolymers and/or the copolymers are preferred. More preferred examples include homopolymers of alpha-hydroxycarboxylic acids; copolymers of two or, more alpha-hydroxycarboxylic acids; and a mixture of the homopolymers and/or the copolymers. Most preferred examples include polylactic acid, copolymer of lactic acid/glycolic acid, copolymer of 2-hydroxybutyric acid/glycolic acid and a mixture thereof.

Alpha-hydroxycarboxylic acids, cyclic dimers of alpha- hydroxycarboxylic acids, hydroxydicarboxylic acids, hydroxytricarboxylic acids can be in D-, L- or D,L-configuration.

When a copolymer of lactic acid/glycolic acid is used as one example of the above polymer, the molar (monomer) ratio is preferably in the approximate range of 100/0 to approximately 50/50 (mole/mole). When a copolymer of 2-hydroxybutyric acid/glycolic acid is used as one example of the above polymer, its molar (monomer) ratio is preferably in the approximate range of 100/0 to approximately 25/75 (mole/mole).

The weight-average molecular weight of the copolymer of lactic acid/glycolic acid is preferably in the approximate range of 3,000 to approximately 30,000 Daltons, more preferably in the approximate range of 5,000 to approximately 20,000 Daltons. When a mixture of a polylactic acid and a copolymer of 2-hydroxybutyric acid/glycolic acid is used as one example of the above polymers, the mixture can be used in a blend ratio in the approximate range of 10/90 to approximately 90/10 (by weight), preferably approximately 25/75 to approximately 75/25 (W/W).

The weight-average molecular weight of the polylactic acid is preferably in the approximate range of 3,000 to approximately 30,000 Daltons, more preferably approximately 5,000 to approximately 20,000 Daltons. A preferred proportion of glycolic acid in a copolymer is in the approximate range of 40 to approximately 70 mole %.

Further, the terminal functionalities or pendant groups of the polymers can be modified, for example, to modify hydrophobicity, hydrophilicity and/or provide, remove or block moieties that can interact with the active agent (via, for example, ionic or hydrogen bonding).

While the invention is described herein primarily in connection with its preferred utility, that is, with respect to micro and nanoparticulate drug substances for use in pharmaceutical compositions, it is also believed to be useful in other applications such as the formulation of particulate cosmetic compositions and the preparation of particulate dispersions for use in image and magnetic recording elements.

Excipients and Adjuvants

Excipients and adjuvants that can be used in the present invention, while potentially having some activity in their own right, are generally defined for this application as compounds that enhance the efficiency and/or efficacy of the effective ingredients, or active agent. It is therefore possible to have more than one effective ingredient, including having more than one API, in a given solution, so that the particles of the present invention contain more than one effective ingredient.

Non-limiting examples of excipients and adjuvants that can be included in the solutions that are to be spray frozen or spray frozen into liquids in accordance with the present invention include: surfactants, fillers, stabilizers, polymers, protease inhibitors, antioxidants and absorption enhancers. Excipients can be chosen to modify the intended function of the effective ingredient, or active agent, by improving flow, or bio-availability, or to control or delay the release of the effective ingredient. Additional nonlimiting examples include: Span 80, Tween 80, Brij 35, Brij 98, Pluronic, sucroester 7, sucroester II, sucroester 15, sodium lauryl sulfate, oleic acid, laureth-9, laureth-8, lauric acid, vitamin E TPGS, Gelucire 50/13, Gelucire 53/10, Labrafil, dipalmitoyl phosphadityl choline, phosphadityl choline, glycolic acid and salts, deoxycholic acid and salts, sodium fusidate, cyclodextrins, polyethylene glycols, labrasol, polyvinyl alcohols, polyvinyl pyrrolidones and tyloxapol. Using the process of the present invention, the morphology of the effective ingredients can be modified, resulting in highly porous particles, such as microparticles and nanoparticles.

Stabilization of the biologically active agent can be accomplished, for example, by the use of a stabilizing agent. “Stabilizing agent” as that term is used herein, is any agent which binds or interacts in a covalent or non-covalent manner or is included with the biologically active agent. Examples of stabilizing agents that can be used are described are U.S. Pat. Nos. 5,716,644, 5,674,534, 5,654,010, 5,667,808, and 5,711,968, all specifically incorporated herein, in their entirety. For example, a metal cation can be complexed with the biologically active agent, or the biologically active agent can be complexed with a polycationic complexing agent such as protamine, albumin, spermidine and spermine, or associated with a “salting-out” salt.

Active Agents

Non-limiting examples of effective ingredients, or active agents that can be used in the disclosed embodiments of the present invention include pharmaceuticals, peptides, nucleic acids, proteins, antibiotics, gene therapy agents, catalysts, adsorbents, pigments, coatings, personal care products, abrasives, particles for sensors, metals, alloys, ceramics, membrane materials, nutritional substances, anti-cancer agents, as well as, active agents used in the agriculture industries such as fungicides, insecticides, pesticides, herbicides, fertilizers and the like. It will be appreciated that this list is not exhaustive and is for demonstrative purposes only. It will be further appreciated that it is possible for one compound to be included in more than one class of effective ingredients, for example, peptides and pharmaceuticals. Also, in certain embodiments the active agent is preferably water-soluble, although active agents soluble in organic solvents are also contemplated and the invention is not limited to water-soluble active agents.

Examples of pharmaceuticals include, but are not limited to antibiotics; analgesics; anticonvulsants; antipyretics; anti-inflammatories; antitussive expectorants; sedatives; antidiabetics, antifungals, antiepileptics, antineoplastics; antiulcer agents; antiparkinsonian agents, antirheumatics, appetite suppressants, biological response modifiers, cardiovascular, agents, central nervous system stimulants, contraceptive agents, diagnostic agents, dopamine receptor agonists, erectile dysfunction agents, fertility agents, gastrointestinal agents, hormones, immunomodulators; antihypercalcexnia agents, mast cell stabilizers, muscle relaxants, nutritional agents, ophthalmic agents, osteoporosis agents, psychotherapeutic agents, parasympathomimetic agents, parasympatholytic agents, respiratory agent, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, antihyperthyroids, anti-asthmatics, nucleic acids; expression vectors; and antivertigo agents.

Further examples of peptides having biological activities and that can be used in embodiments of the invention disclosed herein, include oligopeptides such as insulin, somatostatin, somatostatin derivatives (see U.S. Pat. Nos. 4,087,390, 4,093,574, 4,100,117 and 4,253,998), growth hormone, prolactin, adrenocorticotropic hormone (ACTH), melanocyte-stimulating hormone (MSH), thyrotropin-releasing hormone (TRH), their salts and derivatives (see JP-A 50-121273 and JP-A 52-116465), thyroid-stimulating hormone (TSH), luteinizing hormone (LH), follicle-stimulation hormone (FSH), vasopressin derivatives, desmopressin (see Folia Endocrinologica Japonica, vol. 54, No. 5, pp. 676-691 (1978)), oxytocin, calcitonin, parothyroid hormone, glucagon, gastrin, secretin, pancreozymin, cholecystokinin, angiotensin, human placental lactogen, human chorionic gonadotropin (HCG), enkephalin, enkephalin derivatives (see U.S. Pat. No. 4,277,394 and EP-A 31,567); and polypeptides such as endorphin, kyotorphin, interferon (alpha-type, beta-type, or gamma-type), interleukin (I to XI), tuftsin, thymopoietin, tymosin, thymosthymlin, thymic hormone factor (THF), serum thymic factor (FTS) and derivatives thereof (see U.S. Pat. No. 4,299,438) and, other thymic factors (see Medicine in Progress, vol. 125, No. -10, pp. 835-843 (1983)); tumor necrosis factor TNF, colony stimulating factor (CSF), motilin, deinorphin, bombesin, neurotensin, caerulein, bradykinin, urokinase, asparaginase, kallikrein, substance P, nerve growth factor, blood coagulation factor VIII and IX, lysozyme chloride, polymyxin B, colistin, gramicidin, bacitracin, protein synthesis-stimulating peptide (see G.B: Patent No. 8,232,082), gastric inhibitory polypeptide (GLP), vasoactive intestinal polypeptide (VIP), platelet-derived growth factor (PDGH), growth hormone-releasing factor (GRF, somatoclinine), bone morphagenetic protein (BMP), epidermal growth hormone (EGF), erythropoietin, lysozomal storage-related enzymes such as alpha galactosidase and alpha glucosidase, and the like.

Examples of antitumor or antineoplastic agents include bleomycin hydrochloride, methotrexate, actinomycin D, mitomycin C, vinblastine sulfate, vincristine sulfate, daunonibicin hydrochloride, adriamynin, neocarzinostatin, cytosine arabinoside; fluorouracil, tetrahydrofuryl-5-fluorouracil, picibanil, lentinan, levamisole, bestatin, azimexon, glycyrrhizin, poly A:U, poly ICLC and the like.

Examples of the above antibiotics include gentamicin, dibekacin, kanendomycin, lividomycin, tobromycin, amikacin, fradiomycin, sisomysin, tetracycline, oxytetracycline, roliteracycline, doxycycline, ampicillin, piperacillin, ticarcillin, cefalotin, cefaloridine, cefotiam, cefsulodin, cefinenoxime, cefmetazole, cefazollin, cefataxim, cefoperazone, ceftizoxime, moxolactame, thienamycin, sulfazecine, azusleonam, salts thereof, and the like.

Examples of the sedative include chlorpromazine, prochloperazine, trifluoperazine, atropine, scopolamine, salts thereof and the like.

Examples of the muscle relaxant include pridinol, tubocurarine, pancuronium and the like.

Examples of the antiepileptic agent include phenytoin, ethosuximide, acetazolamide, chlordiazepoxide and the like.

Examples of the antidepressant include imipramine, clomipramine, onxiptiline, phenelzine and the like.

Examples of the antidiabetic agent include: glymidine, glipizide, phenformin, buformin, metformin and the like.

The pharmaceutical effective ingredients can be used in a variety of application modalities, including oral delivery as tablets, capsules or suspensions; pulmonary and nasal delivery; topical delivery as emulsions, ointments or creams; and parenteral delivery as suspensions, microemulsions or depot.

Particles

In addition, the apparatus, compositions, and methods described herein provide for production of microparticles and nanoparticles for delivery of various active agents, such as pharmaceuticals, proteins, peptides, and other active agents described herein. In one embodiment, spray freezing into liquids (SFL) can be used to provide quicker freezing times, which in turn produce a more uniform distribution of particle sizes, smaller particles, more porous particles, and a more intimate mixing of particle components. The intense atomization and rapid heat transfer resulting from spraying directly into a cryogenic media offers additional advantages that include avoiding ice crystal formation, which can degrade proteins/peptides. Atomization in SFL typically leads to smaller zone sizes of the regions that are frozen leading to a molecular dispersion formed of active agent(s) and/or excipients.

Embodiments of the present invention provide API-containing particles, including protein and peptide particles, having novel features. In particular, the proteins and peptides, described herein, are very small and possess enhanced stability. This enhanced stability is reflected, in part, by the reduction of protein aggregation, or monomer loss. Monomer loss of less than about 20%, 15% or 10% protein aggregation is desirable, preferably less that about 7%, 5%, or 3%, more preferably less than about 2% or 1%, most preferably less than about 0.5%, 0.25%, 0.20%, 0.15%, 0.10%, 0.07% or 0.05%, and perhaps even less.

Moreover, stability can be reflected by the secondary structure of the proteins and peptides. Typically, compositions having higher surface areas experience a greater degree of denaturization. The protein and peptide compositions produced by the present invention, possess enhanced degree of stability, that is, they possess higher surface areas without the degree of denaturization experienced by other methods. This enhanced stability can be characterized by the secondary structure (in part reflected by the α-helix or β-sheet values) of the resulting proteins and peptides.

The α-helix and β-sheet values associated with secondary structural folding can be assessed using Fourier transform infrared spectroscopy (FTIR), for either solid or liquid samples. Protein and peptide compositions of the present invention display, in some embodiments, minimal loss of secondary structure. The protein and peptide compositions produced, according to the invention, have % α-helix loss of less than 30%, preferably less than about 25% or 20%, more preferably less than about 15%, 14%, 13%, 12%, or 11%, most preferably less than about 10%, 9% or 8%, and perhaps even less.

Another feature of the proteins and peptides, of the present invention, is a high surface area. The proteins and peptides to have a surface area of greater than about 1 m²/g, it is desirable for the surface area to be greater than about 5 m²/g, 10 m²/g or 15 m²/g, preferably greater than about 17 m²/g or 19 m²/g, more preferably greater than about 20 m²/g, 21 m²/g, 22 m²/g, 23 m²/g, 24 m²/g, 25 m²/g, 26 m²/g, 27 m²/g, 28 m²/g or 29 m²/g, and most preferably greater than about 30 m²/g, 31 m²/g, 32 m²/g, 33 m²/g, 34 m²/g, 35 m²/g, 36 m²/g, 37 m²/g, 38 m²/g or 39 m²/g and perhaps even greater, such as up to about 50 m²/g, 75 m²/g, 100 m²/g 125 m²/g or even 250 m²/g.

Formulation and Administration

A particle preparation of the present invention can be administered to living bodies as a powder formulation, or by molding them in the form of various preparations. Further, a microparticle preparation can be used as raw materials in the production of various preparations including fluid, liquid or cream preparations as well as solid or dry preparations. Pharmaceutical effective ingredients can be used in a variety of application modalities, including but not limited to oral delivery as tablets, capsules or suspensions; pulmonary and nasal delivery; topical delivery as emulsions, ointments or creams; parenteral delivery as suspensions, microemulsions or depot; intrathecal, intraoccular, intramuscular (including as depot) and other delivery methods are also contemplated. Various routes of administration are known to one of skill in the art and additional modes of administration would be applicable in light of the description herein.

In one embodiment the particles containing an active agent(s) can be included in a transdermal device and can include, in its most basic embodiment, a reservoir adapted to retain during storage and release in operation the microparticles containing an active agents(s) produced using the present invention. It will be appreciated that a wide variety of transdermal devices have been described in the art and are suitable for use in the present invention.

An exemplary transdermal device generally includes a reservoir defined by an impermeable backing layer and a membrane. The backing layer and the membrane are joined together about the outer periphery of the device. These layers can be joined by an adhesive, a heat seal or the like. The transdermal device can also include an adhesive layer to attach the device to the skin of a subject. A release liner will generally cover the adhesive that the user removes prior to use of the device.

Formulations of a microparticle composition include, but are not limited to parenteral preparation (for example, injectable preparation), topical preparation (for example, nasal preparation, dermatological preparation, or the like), suppositories (for example, rectal, vaginal), or the like, oral preparation (for example, powders, granules, capsules, tablets, or the like), inhalants, and the like. The amount of drug to be included in the preparations depends on the kind of active agent, dosage form, object of treatment, and so on, of which one skilled in the art would be capable of determining.

The particles containing the active compound(s) can be administered with or without a propellant into, for example, the deep lung or even orally. When administered orally the particles can be provided with, for example, an inert diluent or with an assimilable edible carrier, or they can be enclosed in hard or soft shell gelatin capsule, or they can be compressed into tablets, or they can be incorporated directly with the food of the diet.

For oral therapeutic administration, the particles containing the active compound(s) can be incorporated with excipients and used in the form of ingestible tablets, buccal tablets, troches, capsules, elixirs, suspensions, syrups, wafers. Such compositions and preparations should contain at least 0.1 percent of active compound. The percentage of the compositions and preparations may, of course, be varied and can conveniently be between 2 to 60 percent of the weight of the unit. The amount of particles containing the active compound(s) in such therapeutically useful compositions is such that a suitable dosage will be obtained.

The present invention finds particular uses in the delivery of particles of low density and large size for drug delivery to the pulmonary system. Inhaled aerosols have been used for the treatment of local lung disorders including asthma and cystic fibrosis and have potential for the systemic delivery of peptides and proteins. The particles containing the active compound(s) of the present invention can be used with local and systemic inhalation therapies to provide controlled release of the therapeutic agent.

The particles containing the active compound(s) provided herein permit for an effective dry-powder inhalation therapy for both short and long term release of therapeutics, either for local or systemic delivery, with minimum aggregation. While not wishing to be bound by theory, the increased particle size consistency is expected to decrease the particles' clearance by the lung's natural mechanisms until drugs have been effectively delivered.

As used herein, “pharmaceutically acceptable carrier” includes any and all solvents, dispersion media, coatings, antibacterial and antifungal agents, isotonic and absorption delaying agents. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

Pharmaceutical preparations of the present invention and modifications thereof can be manufactured by using known methods in the field of pharmaceutics. When the microparticle preparations according to the present invention are to be processed into an injectable preparation, they can be dispersed in an aqueous vehicle together with a dispersing agent; for example, Tween 80 (Astrapowder Co., U.S.A.), HCO 60 (Nikko Chemicals, Japan), carboxymethylcellulose, sodium alginate, or the like, preservative (for example, methylparaben, propylparaben, benzyl alcohol, chlorobutariol, or the like), isotonizing agent (for example, sodium chloride, glycerin, sorbitol, glucose, or the like), or the like. The vehicle can also be a vegetable oil (for example, olive oil, sesame oil, peanut oil, cottonseed oil, corn oil, or the like), propylene glycol or the like. In this manner, an injectable preparation can be produced.

When the microparticle preparations according to the present invention are to be processed into oral preparation, they are mixed with an excipient (for example, lactose, sucrose, starch, or the like), disintegrating agent (for example, starch, calcium carbonate, or the like), binder (for example, starch, gum arabic, carboxymethylcellulose, polyvinylpyrrolidone, hydroxypropylcellulose, or the like) and/or lubricant (for example, talc, magnesium stearate, polyethyleneglycol 6000, or the like), and the mixtures are compressed in molds, and then if necessary, the preparations can be coated by known methods for the purpose of masking taste or providing them with enteric or sustained release properties. Usable as coating agents are, for example, hydroxypropylmethylcellulose, ethylcellulose, hydroxymethylcellulose, hydroxypropylcellulose, polyoxyethlene glycol, tween 80, Pluomic, F86, cellulose acetate phthalate, hydroxypropylmethylcellulose phthalate, hydroxymethylcellulose acetate succinate, Eudragit (Roehm, Germany; methacrylic acid-acrylic acid copolymer) and pigments (for example, titanium oxide, ferric oxide, or the like).

To manufacture a topical preparation from the microparticle preparations according to the present invention, they are provided in a solid, semi-solid, or liquid state in the conventional manner. To manufacture the solid topical preparation for instance, the microparticle preparations either as they are or together with an excipient (for example, glucose, mannitol, starch, microcrystalline cellulose, or the like) and/or thickener (for example, natural mucilages, cellulose derivatives, polyacrylates, or the like) are processed into powdery composition.

To manufacture a liquid composition, a microparticle preparation can be processed into an oily or aqueous suspension in substantially the same manner as in the case of injections. The semi-solid preparation can be an aqueous or oily gel or ointment. In any case, there can be added a pH adjusting agent (for example, carbonic acid, phosphoric acid, citric acid, hydrochloric acid, sodium hydroxide, or the like), a preservative (for example, p-hydroxybenzoic acid esters, chlorobutanol, benzalkonium chloride), or the like.

A suppository of a microparticle preparation according to this invention, whether in oily or aqueous solid or semi-solid state or in liquid state, can be produced in a conventional manner. The kind of oleagenous base for such composition is optional as long as it will not dissolve the microparticle preparation. Thus, for example, higher fatty acid glycerides for example, cacao butter, Witepsol (Dynamit-Novel, Germany), or the like, intermediate fatty acids for example, Miglyol (DynamitNovel), or the like, and vegetable oils (for example, sesame oil, soybean oil, cottonseed oil, or the like) can be mentioned. The aqueous base is exemplified by polyethylene glycol and propylene glycol, while the aqueous gel base can be selected from among natural mucilages, cellulose derivatives, vinyl polymers, polyacrylates, or the like

The following examples are for illustrative purposes only and are not intended to limit the scope of the claimed invention. Indeed, various modifications of the described examples which are obvious to those skilled in the art or related arts are intended to be within the scope of the present invention. Percentages are in weight percents unless otherwise stated.

### EXAMPLES

The following terms are used in the subsequent examples.

“Danazol” is a poorly water soluble (aqueous solubility=0.17-0.22 μtg/ml) glucocorticoid derivative that is used commercially in the treatment of breast cancer and endometriosis.

“THF” is tetrahydrofuran.

“HPβCD” is hydroxypropyl-beta-cyclodextrin.

“SFL” means spray freezing into liquid and refers to the process of the present invention.

“XRD” means x-ray diffraction.

“Slowly frozen control” refers to a sample made by placing the drug solution on the tray of a lyophilizer and then slowly cooling the tray to freeze the sample. The ice is then sublimed.

“Co-ground physical mixture” refers to a sample prepared by admixing the active with the excipients in a ceramic mortar, then mixing with a pestle to provide intimate contact and particle size reduction of the powders.

“ATMFD” means atmospheric freeze-drying.

“FeSSIF” means fed state simulated intestinal fluid prepared by a recipe known in the art. This media is used to simulate gastrointestinal conditions. FeSSIF is the dissolution media that the powders produced in the following examples are dispersed into to quantitate the amount of drug dissolved as a function of time.

“CBZ” refers to carbamazepine, a well-established drug used in the treatment of epilepsy, having an aqueous solubility of 61 μg/mL.

“SLS” refers to sodium lauryl sulfate.

“TAA” means trianicinolone acetonide, a long-acting synthetic glucorticoid, which is poorly water soluble (aqueous solubility=21 μg/mL.

“PVP K-15” means polyvinyl pyrrolidone with an approximate molecular weight of 8000.

“Poloxamer 407” means polyoxyethylene-polyoxypropylene copolymer with an average molecular weight distribution of 9840 to 14600

“sCT” means salmon calcitonin.

### Example 1

An aliquot of 0.1 g bovine insulin was dissolved in 20 ml HCl (pH=2), the solution was then titrated by 0.1 N NaOH to pH 7.4. The feed solution was then atomized using an ISCO pump by spray freezing into liquid nitrogen. The nozzle used was polyetheretherketone tubing with a diameter of 63.5 μm. The flow rate was 14 ml/min at 4000 psi. The frozen particles were collected and dried in a tray lyophilizer. The products were characterized by different techniques, and the results are shown in Table 3. The resulting particles were highly porous. The SFL process did not influence the chemical stability of insulin as indicated by no change in the level of A-21 desamido insulin (Table 3). The SFL process decreased the level of covalent insulin dimer (Table 3). The mean particle size diameter as determined by time-of-flight measurement was 5.06 microns. SEM photographs reveal that the particle is an aggregate composed of small domains of insulin.

TABLE 3
The physical and chemical characteristics of SFL insulin powder
compared to bulk insulin powder.

| | A-21 | | | |
|---|---|---|---|---|
| | desamido | Covalent Insulin | BET surface | Mean Particle |
| | insulin | Dimer | area | Size |
| | (percent) | (percent) | (m²/g) | (μm) |
| SFL | 3.04 | 0.36 | 30.16 | 5.06 |
| insulin | | | | |
| powder | | | | |
| Bulk | 3.03 | 0.25 | | 14.24 |
| insulin | | | | |

### Example 2

The SFL insulin powder was prepared according to the procedure outlined in Example 1 with same formulation. The SFL insulin powder was stored at −4° C. The samples were taken out at 6, 12 and 24 weeks. SEM photographs showed that the particles were still porous after 24 weeks storage. The percentage of A-21 desamido and covalent insulin dimer in the sample are summarized in Tables 4 and 5.

TABLE 4
The percentage of A-21 desamido in the samples that was stored
for different time periods.

| | Initial | 6 weeks | 12 weeks | 24 weeks |
|---|---|---|---|---|
| SFL insulin | 2.45 | 2.72 | 3.04 | 3.03 |
| powder | | | | |
| Bulk insulin | 2.46 | | 2.61 | 2.72 |

TABLE 5
The percentage of covalent insulin dimer in the samples
which were stored for different time periods.

| | Initial | 6 weeks | 12 weeks | 24 weeks |
|---|---|---|---|---|
| SFL insulin | 0.5 | 0.55 | 1.47 | 1.25 |
| powder | | | | |
| Bulk insulin | 0.5 | | 1.18 | 1.15 |

### Example 3

Aliquots of 50 mg sCT, 10 mg tyloxapol, and 250 mg lactose were dissolved in 40 ml of deionized water to make the SFL feed solution. Then the feed solution was pressurized using an ISCO syringe pump and atomized by spray freezing into liquid nitrogen. The nozzle used was polyetheretherketone tubing with inner diameter of 63.5 μm. The flow rate was 14 ml/min at a 4000 psi. The frozen particles were collected and dried in a tray lyophilizer. The physical properties are summarized in the Table 6.

TABLE 6
The physical characteristics of SFL calcitonin powder
which was sprayed with tyloxapol and lactose

| | BET surface area | Mean Particle Diameter |
|---|---|---|
| | (m²/g) | (μm) |
| SFL calcitonin powder | 19.16 | 10.49 |

### Example 4

An aliquot of 0.085 g danazol was dissolved in 14.85 g THF, and 0.365 g HPβCD was dissolved in purified water. The two solutions were mixed and allowed to equilibrate to generate a water-soluble inclusion complex between danazol and HPβCD. The solution was then processed by SFL and freeze-dried in a tray lyophilizer. The solution was atomized directly into liquid nitrogen through an insulated polyetheretherketone (PEEK) nozzle with a 63.5 μm orifice at 5000 psi constant pressure supplied by an ISCO syringe pump. The atomized frozen powder was then collected by sieve filtration and freeze-dried. XRD showed that danazol existed in the substantially amorphous form in the engineered powder. The amorphous nature of danazol in the XRD pattern also verifies that the danazol was included into the HPβCD cavity during equilibration of the solution. The BET specific surface area of the SFL powder (113.50 m2/g) is significantly greater than that of the slowly frozen control (0.17 m2/g), co-ground physical mixture (0.42 m2/g), or bulk danazol (0.52 m2/g). The high surface area of the micronized SFL powder can be attributed to the porous nature of the microparticles that compose the engineered powder. The amount of danazol dissolved from the micronized SFL powder within 2 minutes (100 percent) was significantly greater than that from the slowly frozen control (70 percent) or co-ground physical mixture (20 percent). Measurement of the contact angle of compacts of the various samples investigated allowed for the determination of the wettability of the powders. From Table 7, it can be seen that the micronized SFL powders had significantly lower contact angles (27.0-32.3°) with aqueous dissolution media than did the slowly frozen control (32.0°), the coground physical mixture (51.5°) or the danazol alone (64.3°). A lower contact angle coincided with enhanced wettability of the powder in aqueous dissolution media.

TABLE 7
Determination of contact angle determined against purified water.

| Sample | Average Contact Angle ± S.D. |
|---|---|
| SFL Danazol Alone | 64.3 ± 1.1 |
| Co-grounded Physical Mixture | 51.5 ± 0.7 |
| Slowly Frozen Control | 32.0 ± 0.0 |
| Micronized SFL Powder | 27.0 ± 2.8 |

### Example 5

An aliquot of 2.5 g danazol was dissolved in 372.5 g THF. Aliquots of 1.25 g polyvinyl alcohol (PVA, MW=22,000), 1.25 g polyvinylpyrrolidone (PVP) K-15, and 1.25 g poloxamer 407 were dissolved in purified water. The two solutions were mixed to form a one-phase cosolvent solution. The solution was then processed by SFL and freeze-dried and fluidized in an atmospheric freeze-drying (ATMFD) chamber. The solution was atomized directly into liquid nitrogen through an insulated polyetheretherketone (PEEK) nozzle with a 127 μm orifice at 5000 psi constant pressure, which was supplied by an HPLC pump. The atomized frozen powder was then collected by sieve filtration and freeze-dried using a cryogenic atmospheric (ATMFD) fluidized bed apparatus. XRD showed that danazol existed in the substantially amorphous form in micronized SFL powders dried by ATMFD or tray lyophilization. In contrast, crystalline danazol was present in the co-ground physical mixture and slowly frozen control. The specific surface area of the ATMFD-micronized SFL powder (5.72 m2/g) is significantly less than that of the tray lyophilized micronized SFL powder (8.90 m2/g), but significantly greater than that of the co-ground physical mixture (1.92 m2/g) or bulk danazol (0.52 m2/g). The microparticles are porous and have a high surface area. The amount of danazol dissolved from the tray lyophilized micronized SFL powder was 93 percent after 2 minutes whereas the amount dissolved from the ATMFD micronized SFL powder was 79 percent within the same time period. By 5 minutes, danazol was completely dissolved from both micronized SFL powders, regardless of the drying technique utilized. The amount of danazol dissolved from the micronized SFL powders was significantly greater than that from the slowly frozen control, co-ground physical mixture or bulk danazol for the first 20 minutes of the dissolution study. Measurement of the contact angle of compacts of the various samples investigated allowed for the determination of the wettability of the powders. From Table 8, it can be seen that the micronized SFL powders had significantly lower contact angles (48.9° for tray lyophilized and 27.5° for ATMFD) with aqueous dissolution media than did the slowly frozen control (55.0°), the co-ground physical mixture (51.5°) or the danazol alone (64.3°). A lower contact angle coincided with a better wettability of the powder in aqueous dissolution media. Thus, the SFL process produced a high surface area, micronized powders containing amorphous danazol dispersed within a polymeric matrix that optimized the aqueous dissolution of an otherwise poorly water soluble API. The ATMFD process is a suitable alternative to tray lyophilization, and ATMFD does not allow for recrystallization of danazol and produces micronized SFL powders with similar dissolution profiles to those dried by tray lyophilization.

TABLE 8
Investigation of
wettability by determination of contact angle against purified water

| | Average |
|---|---|
| Sample | Contact Angle ± S.D. |
| SFL Danazol Alone | 64.3 ± 1.1 |
| Co-grounded Physical Mixture | 51.5 ± 0.7 |
| Slowly Frozen Control | 55.0 ± 0.42 |
| Tray Lyophilized Micronized SFL Powder | 48.9 ± 2.8 |
| ATMFD Micronized SFL Powder | 27.5 ± 10.5 |

### Example 6

An aliquot of 1.0 g danazol was dissolved in 32.33 g ethyl acetate. Aliquots of 0.5 g polyvinyl alcohol (PVA, MW=22,000), 0.5 g polyvinylpyrrolidone (PVP) K-15, and 0.5 g poloxamer 407 were dissolved in purified water. The organic solution was added drop-wise to the aqueous solution as it was being mixed using a rotor/stator type homogenizer. The resulting o/w emulsion was then further processed using a high-pressure homogenizer. The emulsion was then processed by SFL and tray lyophilized. The emulsion was atomized directly into liquid nitrogen through an insulated polyetheretherketone (PEEK) nozzle with a 127 μm orifice at 5000 psi constant pressure, which was supplied by an ISCO syringe pump. The atomized frozen powder was then collected by sieve filtration and freeze-dried. XRD showed that the resulting danazol existed in the substantially amorphous form in the micronized SFL powder from emulsion and solution. In contrast, crystalline danazol was present in the coground physical mixture. SFL processing of the emulsion produces spherical microparticles about 10-25 μm in diameter. The amount of danazol dissolved from the micronized SFL powder from emulsion was 93 percent after 2 minutes, which is similar to what was dissolved from the SFL powder from solution (93 percent) within the same time period. By 5 minutes, danazol was completely dissolved from both micronized SFL powders, emulsion and solution-based.

### Example 7

An aliquot of 100 mg danazol was dissolved in 14.9 g tetrahydrofuran (THF). An aliquot of 200 mg polyoxyethylene-polyoxypropylene copolymer (pluronic F127; poloxamer 407) was dissolved in the 29.8 g purified water. The aqueous and organic solutions were then mixed to obtain danazol/poloxamer 407 (1:2) SFL feed solution. The SFL solution was held in the solution cell. The solution cell was connected to an insulated nozzle which was positioned within the cryogenic liquid when the cryogenic liquid was in the vessel. Liquid nitrogen was used as the cryogenic liquid in this experiment. The nozzle used was polyetheretherketone (PEEK) tubing with a diameter of 63.5 μm. A constant pressure 4000 PSI from the ISCO syringe pump provided a spray flow rate (15 ml/min) for the SFL feed solution. The SFL feed solutions were then sprayed through the nozzle and atomized into small droplets directly into the liquid nitrogen phase. Frozen particles formed instantaneously. The frozen particles were collected and lyophilized by a tray lyophilizer for 48 hrs. The very fine, porous and uniform SFL powder was generated.

XRD indicates that SFL powders containing danazol are substantially amorphous, as compared to highly crystalline bulk danazol.

BET analysis (Table 9) showed that the surface area of the SFL powder was 11.04 m²/g. This result confirmed the highly porous structure of SFL powders. The contact angle measurement (Table 9) demonstrated that the mean value for SFL danazol/poloxamer 407 powder was 34° against purified water and 25° against FeSSIF.

The SFL danazol powders showed the significantly enhanced dissolution rate. The FeSSIF media used for the danazol dissolution studies reportedly simulate in vivo gastrointestinal fluid; low levels of surfactants were recommended to be included in the dissolution media to give a better correlation between in vitro and in vivo data. The rate of dissolution of micronized bulk danazol was slow; only 21 percent of the danazol dissolved in 60 minutes. The dissolution of the SFL danazol/poloxamer 407 was very fast, the amount dissolved danazol reached 99 percent in only 10 minutes.

TABLE 9
The surface
area and contact angle of SFL danazol/poloxamer 407 powders

| Sample | SFL Danazon/Poloxamer 407 |
|---|---|
| Surface Area (m2/g) | 11.04 |
| Contact Angle Degrees (Purified Water) | 34 |
| Contact Angle Degrees (FeSSIF) | 25 |

### Example 8

CBZ and SLS were dissolved in a THF/water co-solvent system, and then processed using the SFL technology. The SFL feed solution was placed into the solution cell. A constant pressure 4000 PSI from the ISCO syringe pump provided a spray flow rate for the SFL feed solution. The SFL feed solutions were then sprayed through a 63.5 μm diameter PEEK nozzle and atomized into small droplets directly into the liquid nitrogen phase. Frozen particles formed instantaneously. The frozen particles were collected and lyophilized.

The very fine, porous and uniform SFL CBZ/SLS powders were generated. The ratio of CBZ and SLS in the SFL powder is one to one. The particle size distribution for the SFL powders and bulk micronized CBZ (Table 10) were determined in an AERO-DISPERSER based on a time-of-flight measurements. The mean particle diameter of the SFL CBZ/SLS powders was 7.11 μm, which is significantly decreased compared to 39.70 μm of the CBZ starting materials. The span index is used to described the polydispersity in a given particle size distribution and is defined as (D90-D10)/D50, where D10, D50, and D90 are the respective particle size at 10, 50, 90 percent cumulative percent undersize. The span index of SFL CBZ/SLS was 1.31 indicating polydispersity. The contact angle measurement (Table 11) demonstrated that the mean value for the SFL CBZ/SLS powders was 24° against the purified water. BET analysis (Table 11) showed that the surface area of the SFL CBZ/SLS powders was 12.81 m2/g. The dissolved CBZ from the SFL CBZ/SLS powders was 94 percent in only 5 minutes.

TABLE 10
The particle size distribution of SFL
CBZ/SLS powders and bulk micronized CBZ

| | SFL CBZ/SLS | Bulk CBZ |
|---|---|---|
| D10 (μm) | 1.33 | 15.56 |
| D50 (μm) | 7.11 | 39.70 |
| D90 (μm) | 10.61 | 132.33 |
| Span Index | 1.31 | 2.94 |

TABLE 11
The surface area and contact angle of CBZ powders

| Sample | SFL CBZ/SLS Powders |
|---|---|
| Surface Area (m2/g) | 12.81 |
| Contact Angle Degrees (Purified Water) | 24 |

### Example 9

SFL CBZ/PVP K15/poloxamer 407 powders were prepared according to the following procedure. The SFL feed solution was prepared by dissolving 200 mg CBZ and 100 mg poloxamer 407 and 100 mg PVP K-15 in the acetonitrile solvent. The SFL feed solution was then placed into the solution cell. A constant pressure 2000 PSI from the ISCO syringe pump provided a spray flow rate for the SFL feed solution. The SEL feed solutions were then sprayed through a 63.5 pm diameter PEEK nozzle and atomized into small droplets directly into the liquid nitrogen phase. Frozen particles formed instantaneously. The frozen particles were collected and vacuum dried 12 hours. The very fine, porous and uniform SFL CBZ/PVP K-15/poloxamer 407 powders were generated. The ratio of CBZ and excipients in the SFL powder was one to one. Powder XRD analysis indicated the substantially amorphous nature of CBZ. Significant improvements in the dissolution rates were found for SFL CBZ/PVP K-15/poloxamer 407 powders. The amount of CBZ dissolved CBZ from SFL CBZ/PVP K-15/poloxamer 407 powders was 95 percent within 5 minutes.

### Example 10

SFL TAA/PVP K-15/poloxamer 407 powder was generated. The ratio of TAA and excipients in the SFL powder is 1:1. Powder XRD analysis indicated that the TAA was substantially amorphous. 90 percent of the TAA was dissolved at 10 minutes for the SFL powder.

### Example 11

BSA powder was prepared by spray freezing process. An aliquot of 2 g BSA was dissolved in 400 ml deionized water. The feed solution was then pressurized using an ISCO syringe pump and atomized by spray freezing into liquid N₂. The nozzle used was polyetheretherketone (PEEK) tubing with diameter of 63.5 μm. The flow rate was 14 ml/min at 4000 psi. The frozen particles of BSA were collected and the frozen solvent was sublimed in a tray lyophilizer. 400 mg of PLGA (I.V.=0.17) was dissolved in 1 ml of dichloromethane. 100 mg of BSA powder was suspended in 5 ml of dichloromethane and sonicated for 5 minutes. The PLGA polymer solution and BSA suspension were mixed together. The mixture was emulsified into 25 ml of 0.2% PVA aqueous solution. The emulsion was sprayed into liquid N₂ through 127 μm ID PEEK tubing nozzle. The flow rate of spray was 20 ml/min. The frozen PLGA particles containing BSA were collected and dried in a tray lyophilizer. The loading degree was 19.4%.

### Example 12

BSA-Zn complex powder was prepared by spray freezing into liquid process. An aliquot of 2 g BSA was dissolved in 400 ml deionized water, followed by the addition of 0.322 g of Zinc acetate. The feed suspension was then pressurized using an ISCO syringe pump and atomized by spray freezing into liquid N₂. The nozzle used was polyetheretherketone (PEEK) tubing with an inner diameter of 63.5 μm. The flow rate was 14 ml/min at 4000 psi. The frozen particles were collected and dried in a tray lyophilizer. 400 mg of PLGA (I.V.=0.17) was dissolved in 1 ml of dichloromethane. 120 mg of BSA-Zn complex powder was suspended in 5 ml of dichloromethane and sonicated for 5 minutes. The polymer solution and BSA suspension were mixed together. The mixture was emulsified into 25 ml of 0.2% PVA aqueous solution. The emulsion was sprayed into liquid N₂ through a 127 μm ID PEEK tubing nozzle. The flow rate of the spray was 20 ml/min. The frozen PLGA particles containing BSA were collected and dried in a tray lyophilizer. The loading degree was 19.6%.

### Example 13

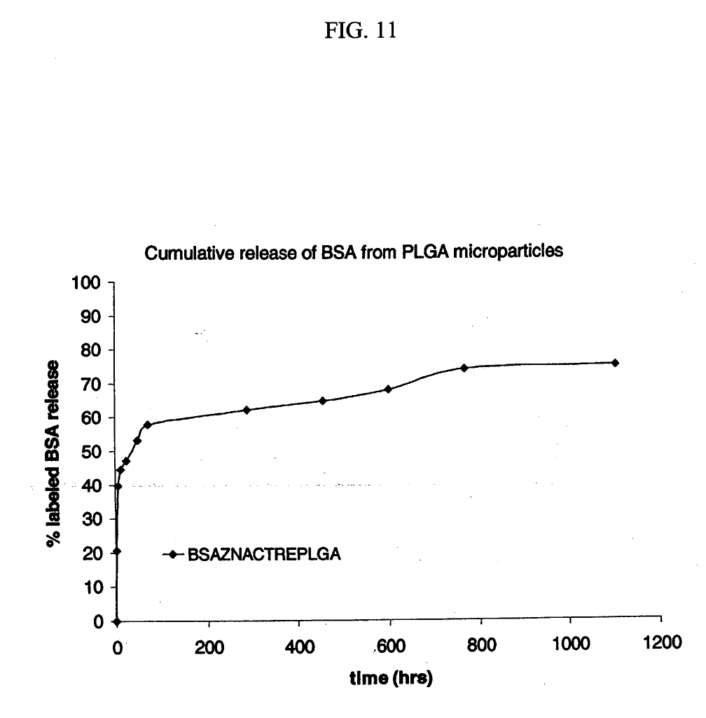

SFL-SO BSA-ZNAC microparticles were prepared in two steps: First, 150 mg BSA was dissolved in 10 mL of 25 mM NaHCO3 buffer solution. To this clear solution was added 1 mL of 2.5% w/v zinc acetate solution to achieve complexation in a molar ratio of 50:1 BSA: ZnAC. 3 mL of a 2.6% w/v trehalose solution was added and the volume of the mixture brought up to 60 mL for a final BSA concentration of 2.5 mg/mL. This solution was then sprayed into liquid nitrogen using the SFL process using a nozzle size of 63.5 μm at a flow rate of 35 mL/min. The frozen particles were collected and then lyophilized in a tray lyophilizer (VirTis Advantage) over 48 hours. Size exclusion chromatography showed a % monomer loss of 0%. In the second step, 109.7 mg of the dried SFL powder containing BSA from step one was incorporated into a 10.36% w/w PLGA (poly lactic-glycolic acid; lactic:glycolide 50:50; intrinsic viscosity 0.59 g/dL; capped) in methylene chloride solution. The resulting suspension was homogenized at 3000 rpm for one minute and then sprayed through a 63.5 um ID nozzle immersed into a layer of liquid nitrogen. This liquid nitrogen layer overlaid a layer of frozen ethanol. The liquid bi-layer containing the frozen particles was stored in a −70° C. freezer for 3 days. Liquid nitrogen evaporates leaving ethanol to extract out methylene chloride from the frozen microparticles. The ethanol containing methylene chloride is poured out and the remainder lyophilized in a tray lyophilizer to obtain a free flowing powder. The final encapsulation efficiency was 85.8% and loading degree was 9.79%. Release studies conducted in 50 mM phosphate buffer solution at 37±0.2° C. using an incubated orbital shaker (Labline instruments) showed a release of 39.6% at 6 hours are illustrated at FIG. 11.

### Example 14

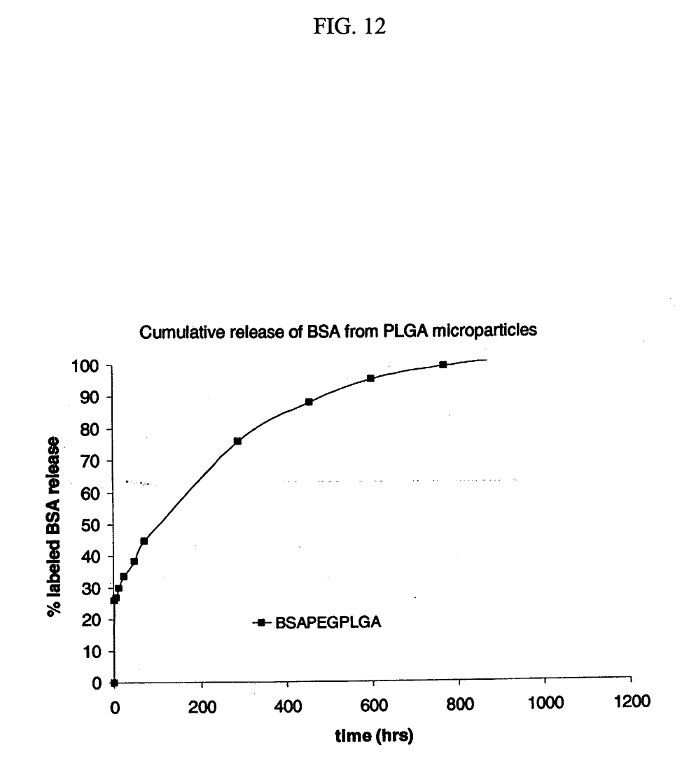

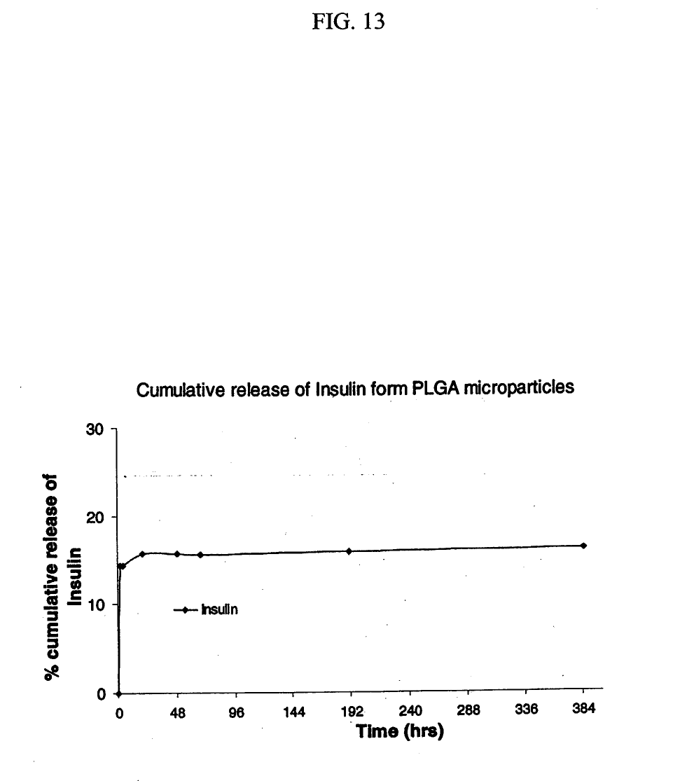

SFL-SO BSA-ZnAC microparticles were synthesized as follows in two steps: First, 150 mg BSA was dissolved in 10 mL 25 mM NaHCO3 buffer solution. To this clear solution was added 4 mL 4.8% w/v PEG 4000 solution. The volume of the mixture was brought up to 60 mL with distilled water for a final BSA concentration of 2.5 mg/mL. This solution was then sprayed into liquid nitrogen thought the SFL apparatus using a nozzle size of 63.5 μm at a flow rate of 35 mL/min. The frozen particles are harvested using a filter and are then lyophilized in a tray lyophilizer (VirTis Advantage) over 48 hours. Size exclusion chromatography showed a % monomer loss of less than 0.5%. In the second step, 132.5 mg of the dried powder from step one was incorporated into a 10.36% w/w PLGA (poly lactic-glycolic acid; lactic:glycolide 50:50; intrinsic viscosity 0.59 g/dL; capped) in methylene chloride solution. The resulting suspension was homogenized at 3000 rpm for one minute and then sprayed through a 63.5 um ID nozzle immersed into a layer of liquid nitrogen. This liquid nitrogen layer overlaid a layer of frozen ethanol. The liquid bi-layer containing the frozen particles was stored in a −70° C. freezer for 3 days. Liquid nitrogen evaporates leaving ethanol to extract out methylene chloride from the frozen microparticles. The ethanol containing methylene chloride is poured out and the remainder lyophilized in a tray lyophilizer to obtain a free flowing powder. The final encapsulation efficiency was 65.7% and loading degree was 7.85%. Release studies conducted in 50 mM phosphate buffer solution at 37±0.2° C. using an incubated orbital shaker (Labline instruments) showed a release of 26.9% at 6 hours are illustrated at FIG. 12.

### Example 15

SFL-SO Insulin microparticles were prepared in two steps: First, 534 mg of insulin was dissolved by incorporating the bulk powder into 10 mL 50 mM Tris (hydroxymethyl) aminomethane buffer solution (pH 7.24). The pH of this dispersion was brought down to acidic range with 0.01N hydrochloric acid solution (HCl) to achieve dissolution and then brought back to a neutral pH of 7.42 using 0.1N sodium hydroxide solution (NaOH). 2.0219 g of trehalose was added as a cryo-protectant. The volume of the mixture was brought up to 60 mL with distilled water for a final insulin concentration of 8.9 mg/mL. This solution was then sprayed into liquid nitrogen using the SFL process using an inner nozzle diameter of 63.5 μm at a constant pressure of 5000 psi. The frozen particles were collected and lyophilized in a tray lyophilizer (VirTis Advantage) over 48 hours. In the second step, 1464 mg of the dried powder from step one was incorporated into a 12.9% w/w PLGA (poly lactic-glycolic acid; lactic: glycolide 50:50; intrinsic viscosity 0.17 g/dL; capped) in methylene chloride solution. The resulting suspension was homogenized at 3000 rpm for one minute and then sprayed through a 63.5 um ID nozzle immersed into a layer of liquid nitrogen. This liquid nitrogen layer overlaid a layer of frozen ethanol. The liquid bi-layer containing the frozen particles was stored in a −70° C. freezer for 3 days. Liquid nitrogen evaporates leaving ethanol to extract out methylene chloride from the frozen microparticles. The ethanol containing methylene chloride is poured out and the remainder lyophilized in a tray lyophilizer to obtain a free flowing powder. The final encapsulation efficiency was 65.7% and loading degree was 4.4%. Stability of insulin was maintained throughout the process as evidenced by no change in the level of degradant product a21 desamido insulin. Release studies conducted in 50 mM phosphate buffer solution at 37±0.2° C. using an incubated orbital shaker (Labline instruments) showed a release of 26.9% at 6 hours are illustrated at FIG. 13.

### Example 16

Bovine insulin powder was prepared by spray freezing process. An aliquot of 0.1 g bovine insulin was dissolved in 20 ml HCl (pH=2.0), the solution was then titrated by 0.1N NaOH to pH 7.4. The feed solution was pressurized using an ISCO syringe pump and atomized by spray freezing into liquid N₂. The nozzle used was polyetheretherketone (PEEK) tubing with diameter of 63.5 μm. The flow rate was 14 ml/min at 4000 psi. The frozen particles were collected and dried in a tray lyophilizes.

The products were characterized by different techniques. SEM photographs showed that the particles were highly porous. Chemical degradation of the SFL processed powder was determined by RP-HPLC and indicated no degradation. The aggregation of insulin in the SFL powder was determined by size exclusion HPLC and no aggregation was found. The median aerodynamic particle size was 5.06 μm.

Following spray freezing, the frozen particles are collected and the solvent is removed. Solvent removal can be accomplished by a variety of techniques, for example lyophilization, or by using a fluidized bed chamber equipped with a stream of gas cooled to a temperature, lower than the melting point of the frozen particles.

A filter-drying unit 200 is depicted in FIG. 2. The freeze-sprayed particles are transferred to the unit 200 so that they reside between two porous filter elements 210. The porous filter elements 210 can be of any material that allows retention of the particles within the unit 200. One example of a suitable porous filter element 210 is a glass frit, but other components can be envisaged by those of skill in the art in light of the present disclosure.

The frozen particles are introduced into the unit 200 via an access joint 240. The unit 200 is attached to a source of cooled gas via inlet 220. The incoming gas will most often be cooled to a temperature below the melting point of the particles so that greatly reduced redissolution takes place. If necessary, the entire unit 200 can be cooled externally, by immersion in a suitable refrigerant. The cooled gas passes through the unit 200 from the bottom to top and passes out of the unit 200 via outlet 230. In this manner the solvent is removed from the frozen particles.

The process can also be operated in a continuous flow mode as depicted in FIG. 3. Similar components are used in the exemplary process of FIG. 1 and like components have retained the number of corresponding components in FIG. 1. By using a continuous flow of the solution source 100 and/or the cryogenic liquid the throughput of the compound that is being spray frozen can be increased dramatically with the incorporation of a filtration unit 300 for the removal of the frozen particles. As will be known to those of skill in the art of pharmaceutical automation, it will be useful that the system is self-contained and capable of being sterilized. In this manner the spray freezing occurs in continuous mode and the microparticles or nanoparticles are captured downstream from the spray-freezing site. The capture mechanism can include a bucket or conveyor belt system that takes the particles and places them in storage, for subsequent processing or for removal of the solvent.

Whether using the batch or the continuous flow method, the size, shape, density, porosity and flowability of the particles can be modified by, for example, varying the solvent, and the relative proportion of any cosolvent (when one is used). It is also possible to alter the morphology of the particles by varying the temperature of the solution in the solution source, the concentration of the solutes, the flow rate of the solution through the nozzle, the inner diameter and/or shape of the nozzle and the pressure drop between the transfer line and the nozzle, to name a few options. Those of skill in the art will recognize that known parameters can be used, with minimal investigation, to obtain, desired particle sizes.

### Example 17

The volume of the internal aqueous phase was 0.3 ml. Insulin concentration in the internal aqueous phase is 154 mg/ml. The Poloxamer 407 Pluronic F-127 concentration in the internal aqueous phase was 50 mg/ml (it functions as protein stabilizer).

The oil phase was 4 ml of methylene chloride. The concentration of poly (D,L-lactideco-glycolide) (DL-PLGA 50:50) was 400 mg/4 ml of oil phase (6.9% w/w). External aqueous phase:

An aqueous solution of 3.5% w/v poly vinyl alcohol (PVA) was used as the external aqueous phase. PVA functions as emulsion stabilizer. The volume of the external aqueous phase was 15 ml.

The resulting w/o/w double emulsion was then sprayed into liquid nitrogen using an ISCO pump via a capillary nozzle with an opening of 127 μm.

Result:

The entrapment efficiency was 83.4% and the loading degree was 8.6%

### Example 18

The volume of the internal aqueous phase was 0.3 ml. Insulin concentration in the internal aqueous phase 165 mg/ml. The Poloxamer 407 Pluronic F-127 concentration in the internal aqueous phase was 25 mg/mi (it functions as protein stabilizer).

The oil phase comprises two different inherent viscosity (I.V.) D,L,-PLGA polymers. The higher the molecular weight the higher the viscosity. The volume of the oil phase was 3.5 ml and it was consisted of 3 ml methylene chloride and 0.5 ml Acetone (Acetone was added in order to improve the solidification rate of the oil phase). The polymer composition was 2:8 (I.V. 0.49:0.17) of two different inherent viscosity of the same type of polymer poly (D,L-lactide-co-glycolide) (D,L-PLGA 50:50). The polymer concentration was 405 mg/3.5 ml organic phase (8.53% w/w).

An aqueous solution of 5% w/v poly vinyl alcohol (PVA) was used as the external aqueous phase. PVA functions as emulsion stabilizer. The volume of the external aqueous phase was 15 ml. The resulting w/o/w double emulsion was then sprayed into liquid nitrogen using an ISCO pump via a capillary nozzle with an opening of 127 μm.

Results:

The entrapment efficiency was 83.8% and the loading degree was 9.1%

### Example 19

An exemplary embodiment of methods described herein is based on a combination of the double emulsion technique and a cryogenic process for production of protein-loaded microparticles is illustrated at FIG. 4. A solution of protein/peptide (internal aqueous phase) was emulsified into the organic phase comprising biodegradable polymer (DL-PLA, L-PLA, DL-PLGA or PCL) dissolved in methylene chloride. The resulting w/o emulsion was added to a solution of polyvinyl alcohol (PVA) and then mixed to obtain the w/o/w double emulsion. The multi-emulsion formulation was subsequently atomized into a cryogenic liquid and frozen. The frozen double emulsion particles were then collected and lyophilized. The dry product was then re-suspended in water and washed three times with distilled water and subsequently lyophilized over night to obtain a water dispersible powder.

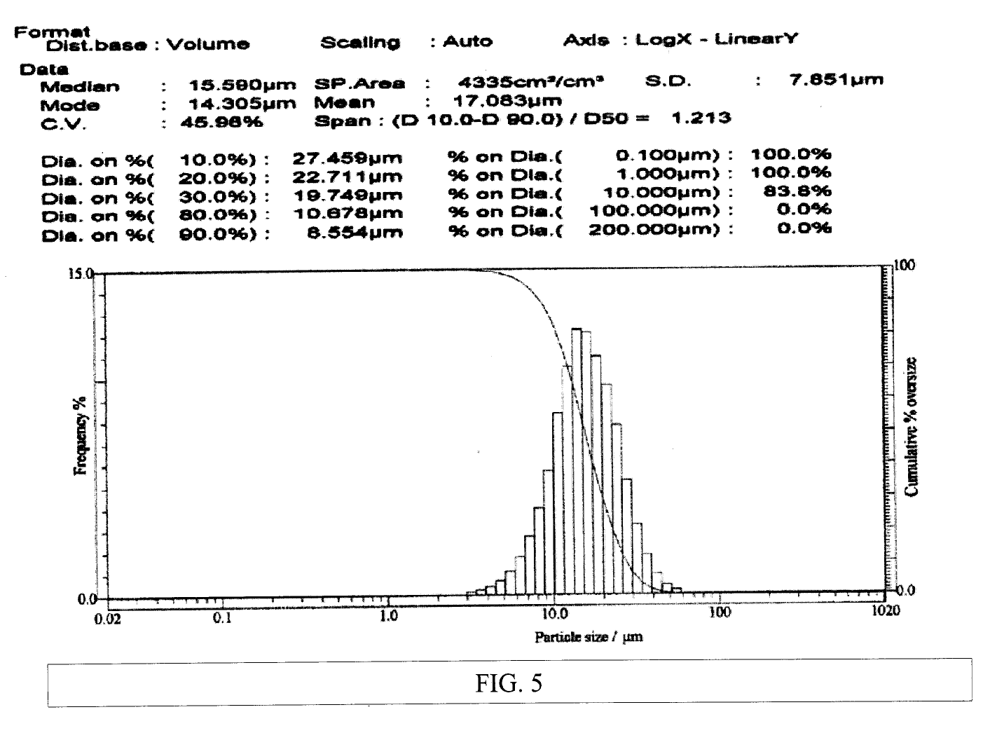

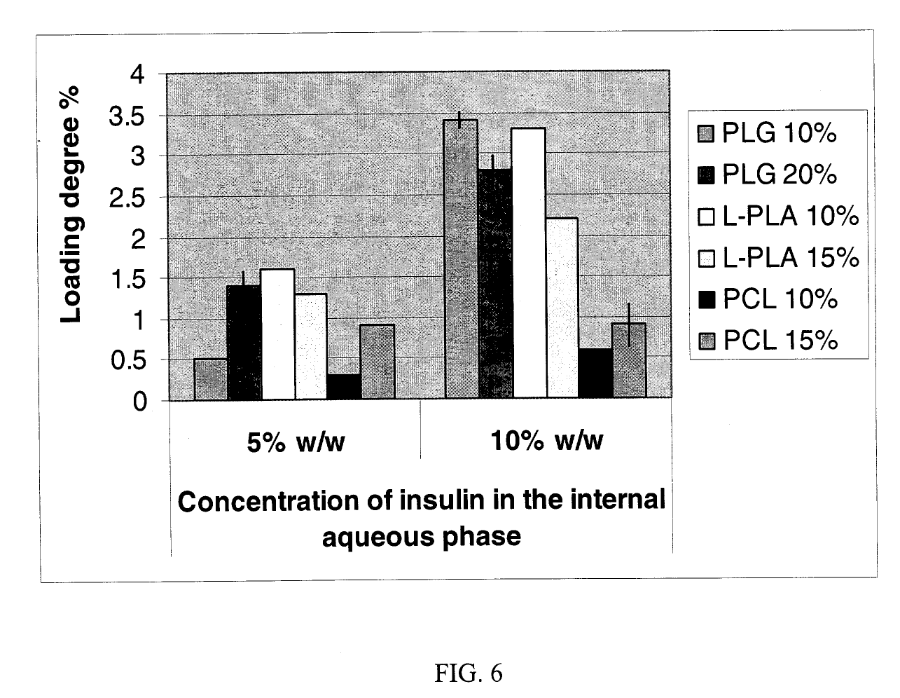

The microparticles obtained were discrete and compact with almost spherical shape. The particle size was between 12-17 μm is illustrated at FIG. 5. The protein/polymer ratio has a strong effect on the entrapment efficiency and loading degree of the incorporated protein. The entrapment efficiency was high for all investigated biodegradable polymers. High polymer concentration yielded high entrapment efficiency for all observed biodegradable polymers and is illustrated at FIG. 6. Scanning Electron Micrographs (SEM) were taken of microparticles prepared by the following conditions: Insulin 8% w/v, Poloxamer 4% w/v in 0.1 M NaOH (internal aqueous phase), DL-PLGA 10% w/w in dichloromethane (oil phase), PVA 3.2% w/v in water (external aqueous phase). An adsorption isotherm derived by S. Brunaure, P. H. Emmett, and E. Teller in 1938 (BET method), as described in “Principals of Colloid and Surface Chemistry”, Second Edition by Paul C. Hiemenz, Marcel Dekeker, Inc., New York and Basel, 1986, (pp. 513-530) was used to determent the specific surface area of 21.37 m²/g on a Nova 2000 high speed gas sorption analyzer (Quantachrom).

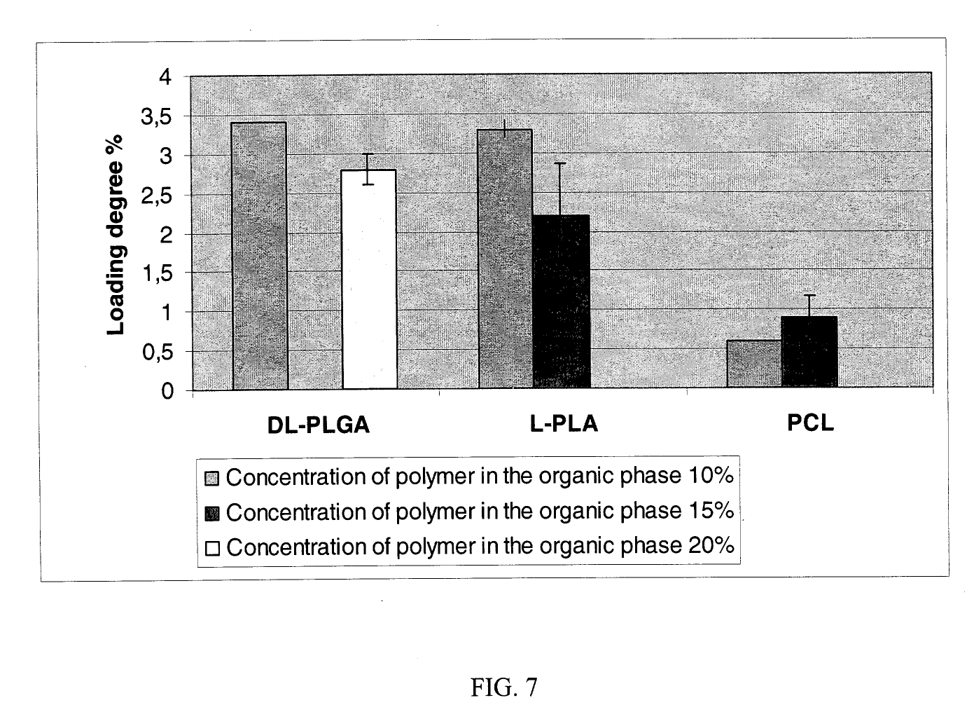

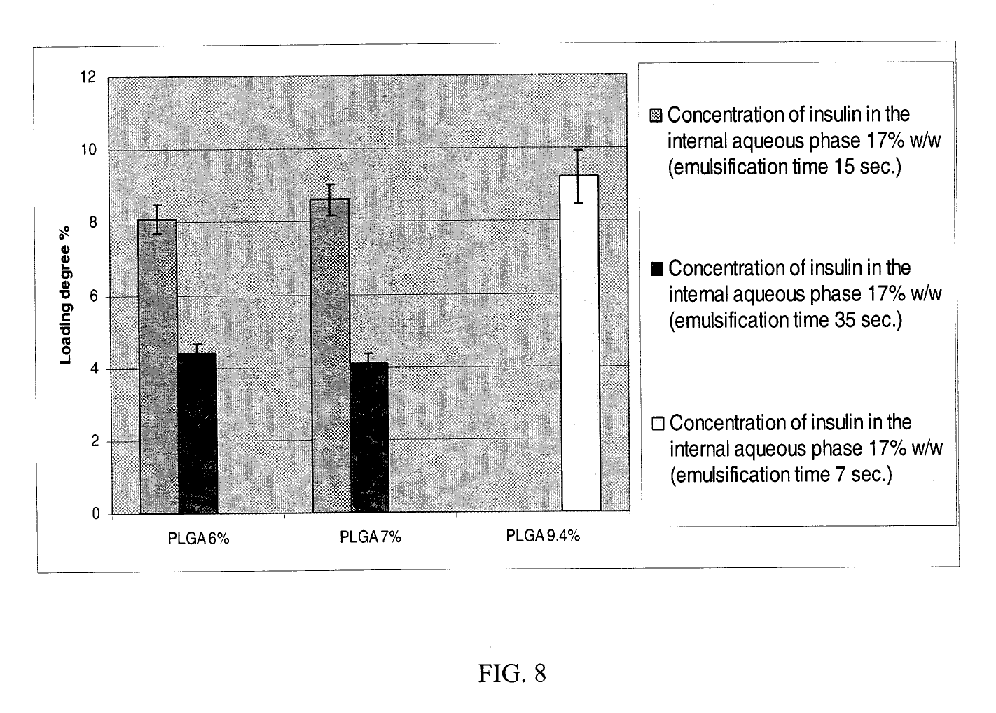

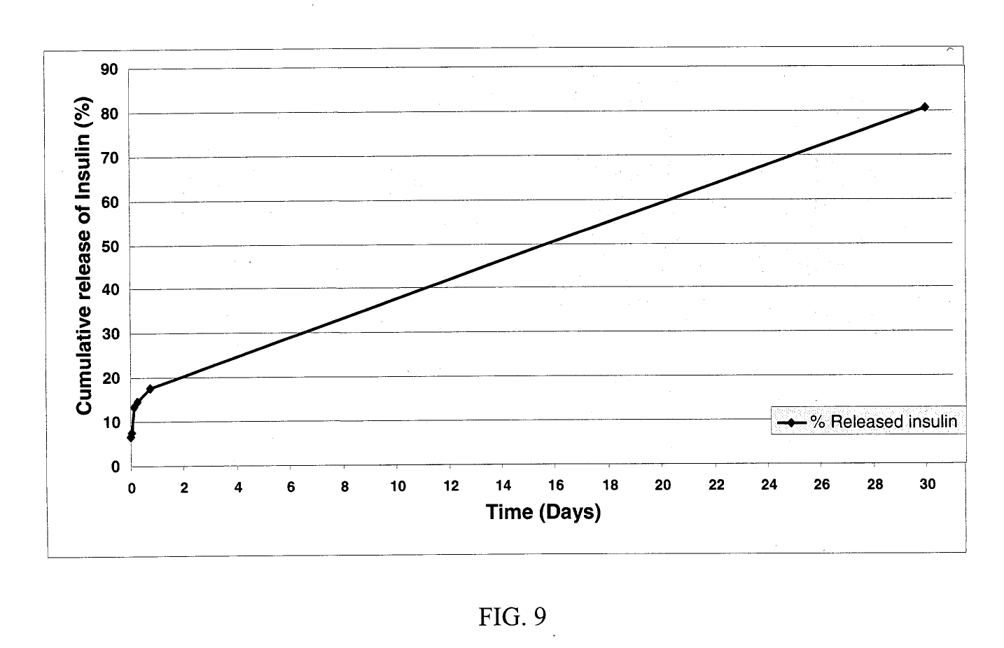

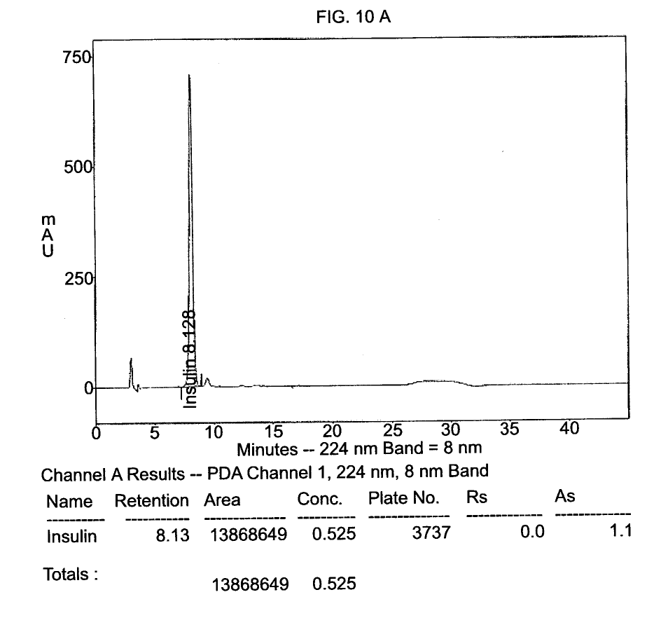

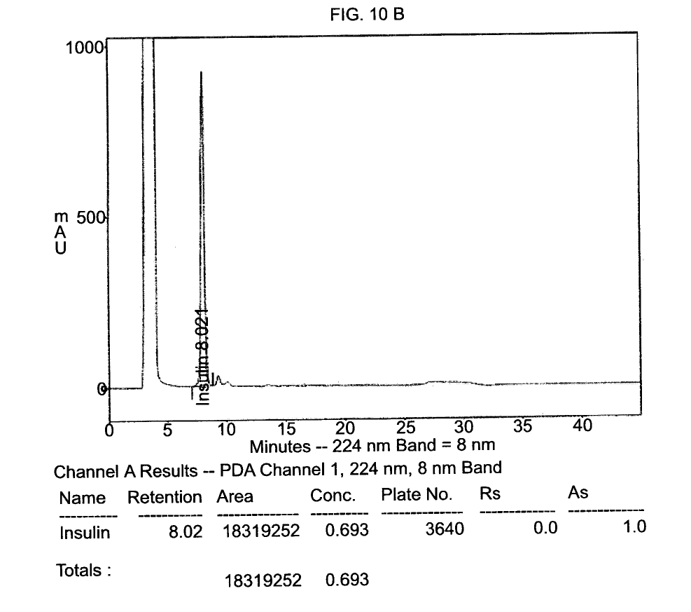

FIG. 7 shows the effect of different types of polymer and polymer concentration within the organic phase on the loading degree of insulin. Entrapment efficiency and loading degree is illustrated at FIG. 8. A release study of insulin showed a low burst effect. The release of insulin from the microparticles was continuous and at a constant rate is illustrated at FIG. 9. The stability of the insulin was maintained throughout the process as evidenced by no change in level of the degradation product A21-desamido insulin are illustrated at FIGS. 10A and 10B.

TABLE 12
Amount Of Insulin Retained Inside The Microparticles
After 30 Days Of Incubation
In Release Media.

| Type and concentration | Retained insulin within the | Number |
|---|---|---|
| of | microparticles after 30 days | of |
| polymer | (% = +/− S.D.) | batches |
| DL-PLGA (20% w/w) | 25.7 | 2 |
| 655 mg/2 ml | | |
| L-PLA (15% w/w) | 29.4 | 1 |
| 462 mg/2 ml | | |
| L-PLA (10% w/w) | 29.2 (4.6) | 3 |
| 291 mg/2 ml | | |

Concentration of Insulin was kept constant at 10% w/w (20 mg/0.2 ml) within the internal aqueous phase for all experiments.

It was demonstrated that the SFL technology can be an optimum method for production of protein/peptide-loaded microparticles, based on biodegradable polymers. The method leads to high entrapment efficiency, high loading degree and low burst effect.

### Example 20

The carbamazepine (CBZ)/ Poloxamer 407/PVP K15 (2:1:1) powder was prepared by the SFL organic solvent system. Aliquots of 200 mg CBZ, 100 mg Poloxamer 407, and 100 mg PVP K-15 were dissolved in 115 ml acetonitrile to make the SFL feed solution and then processed using SFL technology. The SFL feed solution was placed into the solution cell. A constant pressure of 2000 PSI from the ISCO syringe pump provided a spray flow rate (20 ml/min) for the SFL feed solution. The SFL feed solution was then sprayed through the nozzle (63.5 μm) located beneath the surface of the liquid N₂ and atomized into small droplets. The frozen particles were then collected and lyophilized.

The X-ray diffraction pattern indicated that SFL powders containing CBZ was amorphous. Scanning electron microscope (SEM) revealed that SFL CBZ/Poloxamer 407/PVP K15 (2:1:1) powders were porous with a mean particle size of about 500 nm. Surface area of SFL CBZ/Poloxamer 407/PVP K15 (2:1:1) powder was 12.89 m²/g. The contact angle of SFL powders against water was about 35°. Greater than 95% of CBZ/Poloxamer 407/PVP K15 (2:1:1) was dissolved in water within 10 minutes compared to about 4% for bulk micronized CBZ.

### Example 21

The danazol/PEG 8000 (1:2) powder was prepared by the SFL organic solvent system. Aliquots of 200 mg danazol and 400 mg PEG 8000 were dissolved in 70 mL acetonitrile to form the SFL feed solution and then processed using SFL technology. The SFL feed solution was placed into the solution cell. A constant pressure 2000 PSI from the ISCO syringe pump provided a spray flow rate (20 ml/min) for the SFL feed solution. The SFL feed solution was then sprayed through the nozzle (63.5 μm) located beneath the surface of the liquid N₂ and atomized into small droplets. The frozen particles were collected and lyophilized.

The X-ray diffraction pattern indicated that SFL powders containing danazol were amorphous. SEM photographs indicated that SFL danazol/PEG 8000 (1:2) particles were highly porous particles with a mean diameter of about 6 μm. BET analysis showed that the surface area of the SFL powder was 12.0 m²/g. The dissolution of SFL danazol/PEG 8000 K15 (1:2) powder at 5 minutes was 100%.

### Example 22

The danazol/PVP K15 (1:1) powder was prepared by the SFL organic solvent system. Aliquots of 200 mg danazol and 200 mg polyvinylpyrrolidone (PVP) K15 were dissolved in 65 mL acetonitrile and 5 mL of methylene chloride to form the SFL feed solution and then processed using SFL technology. The SFL feed solution was placed into the solution cell. A constant pressure 2000 PSI from the ISCO syringe pump provided a spray flow rate (50 ml/min) for the SFL feed solution. The SFL feed solution was then sprayed through the nozzle (127 μm) located beneath the surface of the liquid N₂ and atomized into small droplets. Frozen particles formed instantaneously. The frozen particles were collected and lyophilized.

The drug potency of the SFL danazol/PVP K15 (1:1) powder was 50%. The X-ray diffraction pattern indicated that SFL powders containing danazol were amorphous. SEM photographs indicated that SFL danazol/PVP K15 (1:1) particles were highly porous particles with a mean diameter of about 750 nm. BET analysis indicated that the surface area of the SFL powder was 115.52 m²/g. This result confirmed the highly porous structure of SFL powders observed by SEM. The contact angle for SFL danazol/PVP K15 powder was 22° against the dissolution media. The amount of SFL danazol/PVP K15 (1:1) powder dissolved at 5 minutes was 100%.

### Example 23

The danazol/PVP K15 (3:1) powder was prepared by the SFL organic solvent system. Aliquots of 600 mg danazol and 200 mg polyvinylpyrrolidone (PVP) K-15 were dissolved in 65 mL acetonitrile and 5 mL of methylene chloride to make the SFL feed solution and then processed using SFL technology. The SFL feed solution was placed into the solution cell. A constant pressure 2000 PSI from the ISCO syringe pump provided a spray flow rate (50 ml/min) for the SFL feed solution. The SFL feed solution was then sprayed through the nozzle (127 μm) located beneath the surface of the liquid N₂ and atomized into small droplets. The frozen particles were collected and lyophilized. The drug potency of the SFL danazol/PVP K15 (3:1) powder was 75%. Very fine, porous danazol/PVP K15 powder was generated. The X-ray diffraction pattern indicated that SFL powder containing danazol was amorphous. SEM photographs indicated that SFL danazol/PVP K15 (3:1) particles were highly porous particles with a mean diameter of about 1.5 μm. BET analysis showed that the surface area of the SFL powder was 68.88 m²/g. The contact angle for SFL danazol/PVP K15 powder was 32° against dissolution media. The dissolution of SFL danazol/PVP K15 (3:1) powder at 5 minutes was 99.4%.

### Example 24

BSA powder was prepared by spray freezing into liquid process. An aliquot of 100 mg BSA was dissolved in 20 ml 5 mM phosphate buffer. The feed solution was then pressured using an ISCO syringe pump and atomized by spray freezing into liquid N₂. The nozzle used was polyetheretherketone (PEEK) tubing with diameter of 63.5 μm. The flow rate was 12 ml/min at 5000 psi. The frozen particles were collected and dried in a tray lyophilizer. The products were characterized by different techniques. Monomer loss of BSA was 0.4%. The specific surface area is 32.3 m²/g. α-helix content of sample was 45.3%, representing a loss of 11.7% compared to BSA prior to SFL processing.

### Example 25

BSA powder was prepared using the same process as Example 24. An aliquot of 100 mg BSA and 20 mg tyloxapol were dissolved in 20 ml 5 mM phosphate buffer. The feed solution was then pressured using an ISCO syringe pump and atomized by spray freezing into liquid N₂. The nozzle used was polyetheretherketone (PEEK) tubing with diameter of 63.5 μm. The flow rate was 12 ml/min at 5000 psi. The frozen particles were collected and dried in a tray lyophilizer. The products were characterized by different techniques. Monomer loss of BSA was 0%. The specific surface area is 27.7 m²/g. α-helix content of sample was 43.1, representing a loss of 16% compared to BSA prior to SFL processing.

### Example 26

BSA powder was prepared using the same process as Example 24. An aliquot of 100 mg BSA and 500 mg trehalose were dissolved in 20 ml 5 mM phosphate buffer. The feed solution was then pressured using an ISCO syringe pump and atomized by spray freezing into liquid N₂. The nozzle used was polyetheretherketone (PEEK) tubing with diameter of 63.5 μm. The flow rate was 12 ml/min at 5000 psi. The frozen particles were collected and dried in a tray lyophilizer. The products were characterized by different techniques. Monomer loss of BSA was 0%. The specific surface area is 19.2 m²/g. α-helix content of sample was 46.2% representing a loss of 9.9% compared to BSA prior to SFL processing.

### Example 27

BSA powder was prepared using the same process as Example 24. An aliquot of 1 g BSA were dissolved in 200 ml deionized water. The feed solution was then pressured using an ISCO syringe pump and atomized by spray freezing into liquid N₂. The nozzle used was polyetheretherketone (PEEK) tubing with diameter of 63.5 μm. The flow rate was 12 ml/min at 5000 psi. The frozen particles were collected and dried in a tray lyophilizer. The products were characterized by different techniques. The specific surface area is 97.7 m²/g. Monomer loss of BSA was 0.8%. The alpha helix content was decreased to about 16%.

### Example 28

BSA powder was prepared using the same process as Example 24. An aliquot of 600 mg BSA were dissolved in 60 ml 5 mM phosphate buffer. The feed solution was then pressured using an ISCO syringe pump and atomized by spray freezing into liquid N₂. The nozzle used was polyetheretherketone (PEEK) tubing with diameter of 63.5 μm. The flow rate was 12 ml/min at 5000 psi. The frozen particles were collected and dried in a tray lyophilizer. The products were characterized by different techniques. The specific surface area is 71.0 m²/g. Monomer loss of BSA was 0.5%. The alpha helix content was decreased to about 13%.

### Example 29

BSA powder was prepared using the same process as Example 24. An aliquot of 120 mg BSA were dissolved in 120 ml 5 mM phosphate buffer. The feed solution was then pressured using an ISCO syringe pump and atomized by spray freezing into liquid N₂. The nozzle used was polyetheretherketone (PEEK) tubing with diameter of 63.5 μm. The flow rate was 12 ml/min at 5000 psi. The frozen particles were collected and dried in a tray lyophilizer. The products were characterized by different techniques. The specific surface area is 23.5 m²/g. Monomer loss of BSA was 0.4%. The alpha helix content was decreased to about 16%.

Although the invention has been described in connection with specific embodiments, it should be understood that the invention as claimed should not be unduly limited to such specific embodiments. Modifications and variations of the described compositions and methods of the invention will be apparent to those skilled in the art without departing from the scope and spirit of the invention. Indeed, various modifications of the described compositions and modes of carrying out the invention which are obvious to those skilled in the art or related arts are intended to be within the scope of the following claims.

### Example 30

Danazol was formulated with polyvinyl alcohol (MW 22,000), poloxamer 407, and polyvinylpyrrolidone K-15 in a 2:1:1:1 weight ratio (40% API potency based on dry weight). Emulsions were formulated in ratios up to 20:1:1:1 (87% API potency based on dry weight). Ethyl acetate/water or dichloromethane/water mixtures were used to produce o/w emulsions for SFL micronization, and a tetrahydrofuran/water mixture was used to formulate the feed solutions. Micronized SFL powders were characterized by x-ray diffraction, surface area, scanning and transmission electron microscopy, contact angle and dissolution (SLS 0.75%/Tris 1.21% media).

Micronized SFL Powder Preparation from Emulsion

The emulsion formulations investigated in the study are shown in Table 13. After determining the saturation solubility of danazol in both EA and DCM, predetermined amounts of danazol were weighed and dissolved in organic solvent. PVA, poloxamer, and PVP were dissolved in water. Water was heated to 80° C. to dissolve the PVA, and then cooled to room temperature to add PVP. The solution temperature was decreased to 0° C. to dissolve the poloxamer, followed by equilibration to room temperature. The oil phase was slowly poured into the aqueous phase under constant mixing, and then blended for 1 minute using a Polytron rotor-and-stator homogenizer (Polytron 10/35 with TS 10 mm Generator, VWR Scientific Corporation, West Chester, Pa.). The emulsion was homogenized for 10 cycles at 20,000 PSI (138 MPa) using an Avestin Emulsiflex-C5 (Avestin, Inc., Ottawa, ON, Canada) high-pressure homogenizer.

TABLE 13
Emulsion formulations that were investigated and processed to obtain micronized SFL powders.

| Formulation | Danazol² | Organic Solvent | PVA | Poloxamer | PVP | Water | Ratio of danazol to excipients by weight |
|---|---|---|---|---|---|---|---|
| Abbreviation | % w/w | % w/w | % w/w | % w/w | % w/w | % w/w | (Danazol:PVA:Poloxamer:PVP) |
| Control¹ | 0.22 | 33.11 THF | 0.11 | 0.11 | 0.11 | 66.33 | 2:1:1:1 |
| A | 1.00 | 32.30 EA³ | 0.50 | 0.50 | 0.50 | 65.20 | 2:1:1:1 |
| B | 1.50 | 50.00 EA | 0.75 | 0.75 | 0.75 | 46.25 | 2:1:1:1 |
| C | 3.00 | 30.00 DCM⁴ | 1.50 | 1.50 | 1.50 | 62.50 | 2:1:1:1 |
| D | 5.00 | 50.00 DCM | 0.50 | 0.50 | 0.50 | 45.50 | 10:1:1:1 |
| E | 5.00 | 50.00 DCM | 0.25 | 0.25 | 0.25 | 44.25 | 20:1:1:1 |

¹The control was a cosolvent solution formulated dissolving danazol in THF and PVA, poloxamer and PVP in water. The two solutions were then mixed to form the control feed solution for SFL processing.
²The percentage of danazol in the total emulsion or control formulation.
³EA is ethyl acetate.
⁴DCM is dichloromethane.

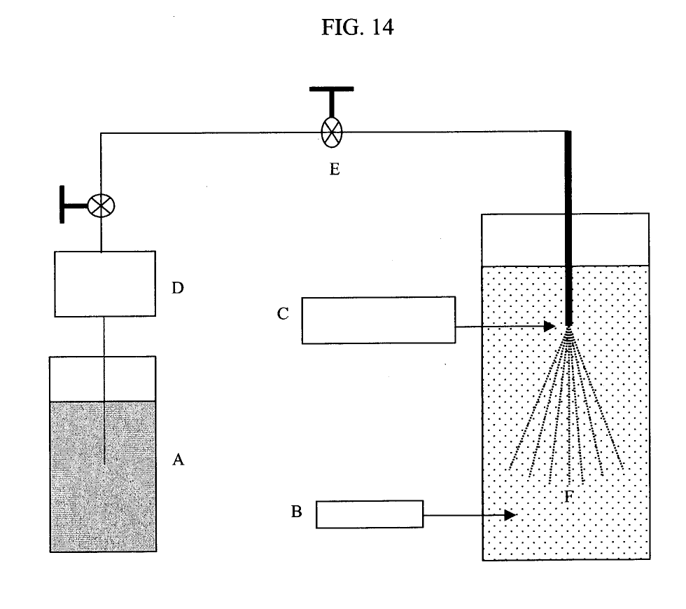

The emulsion formulations were processed by SFL using the apparatus shown in FIG. 14. Each emulsion (FIG. 14a) was atomized beneath the liquid nitrogen surface (FIG. 14b) at 5000 PSI (34.5 MPa) and a flow rate of 20 mL/minute through a 127 μm I.D. polyether-ether ketone (PEEK) nozzle (FIG. 14c) 15 cm in length using an ISCO Model 100DX syringe pump (FIG. 14d; ISCO, Inc., Lincoln, Nebr.). The cryogenic suspension was then poured into a non-insulated beaker to allow the nitrogen to evaporate. Once the nitrogen had evaporated, the frozen micronized SFL powder was immediately vacuum freeze-dried using a VirTis Advantage Benchtop Tray Lyophilizer (The VirTis Company, Inc. Gardiner, N.Y.) with a liquid nitrogen trap. A vacuum of 100 mTorr was maintained throughout the lyophilization period. The shelf temperature was equilibrated to −40° C. The frozen SFL samples were placed in the lyophilizer, and the shelf temperature was increased to +25° C. over the duration of the lyophilization cycle at a rate of 0.9° C. per minute.

Preparation of Control Formulations

A co-ground physical mixture consisting of 1.0 g danazol, 0.5 g PVA, 0.5 g poloxamer and 0.5 g PVP (2:1:1:1 weight ratio) was mixed by geometric dilution and ground using a mortar and pestle for 10 minutes, then mixed for 30 minutes in a V-blender.

A micronized SFL powder from solution was prepared at a 2:1:1:1 (danazol:PVA:poloxamer:PVP) weight ratio and utilized as a control following an earlier procedure. An aliquot of 0.11 g danazol was dissolved in 16.56 g THF. Aliquots of 0.06 g PVA, 0.06 g poloxamer, and 0.06 g PVP were dissolved in 33.17 g purified water. The two solutions were added together and mixed to form a one-phase cosolvent solution. The cosolvent solution was then processed by SFL.

This example shows that emulsions processed by SFL produced micronized powders containing amorphous drug. The surface area increased as drug and excipient concentrations were increased. Surface areas ranged from 8.9 m²/g (SFL powder from solution) to 83.1 m²/g (SFL powder from emulsion). Danazol contained in micronized SFL powders from emulsion and solution was 100% dissolved in the dissolution media within 2 minutes, which was significantly faster than the dissolution of non-SFL processed controls investigated (<50% in 2 minutes). It was demonstrated that micronized SFL powders produced from emulsion had similar dissolution enhancement compared to those produced from solution, but higher quantities (up to 25 times) could be SFL processed from emulsions. The API:excipient ratios in the SFL powder were increased to as high as 87% API potency based on dry weight to achieve rapid wetting and dissolution when utilizing feed emulsions instead of solutions.

Micronized SFL Powders from Emulsions

The emulsion formulations that were SFL processed to micronized powders are shown in Table 13. The equilibrium solubilities of danazol in EA and DCM were 50 mg/mL and 162 mg/ml, respectively. Therefore, emulsion formulations containing 1 and 1.5% danazol (% w/w of total formulation) in EA as the internal oil phase, and emulsions containing 3 and 5% danazol in DCM as the internal oil phase were prepared.

The PSDs of the dry micronized SFL powders from emulsion are listed in Table 14. The M50 of bulk danazol was 23.10 μm. The M50 values were lower for the micronized SFL A (8.16 μm) and SFL D (4.15 μm) powders than the other powders investigated (p<0.05). The span index ranged between 1.55 and 3.98 for the micronized SFL powders investigated, and the magnitude was influenced by composition.

The DSC studies showed that poloxamer and PVP melted at 50° C., and PVA melted at 80° C. Bulk danazol melted at 225° C. All micronized SFL powders from emulsion exhibited similar DSC profiles to the co-ground physical mixture (data not shown). Poloxamer/PVP and PVA melting endotherms were observed at 50 and 80° C., respectively; and a danazol crystallization exotherm occurred at 200° C.

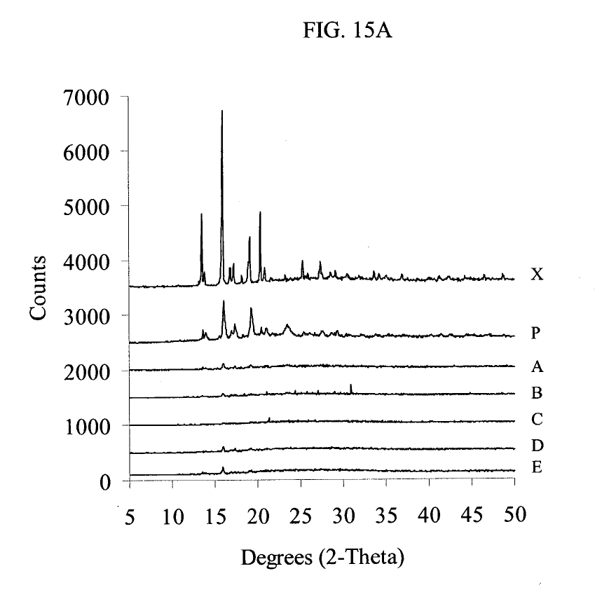

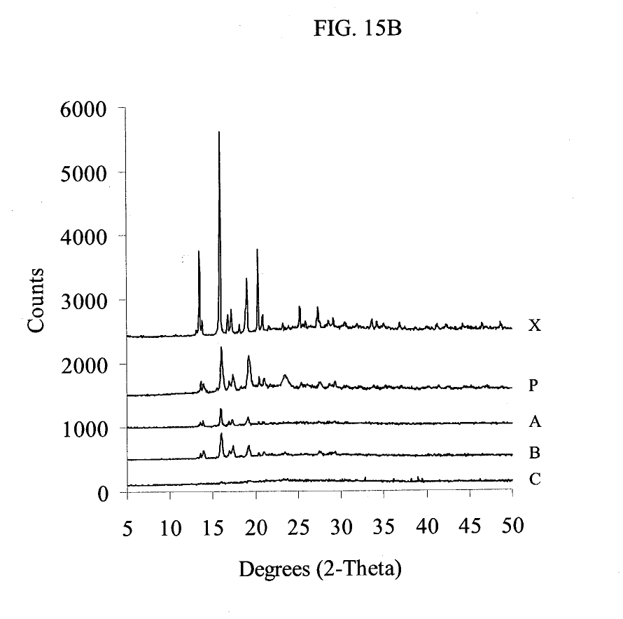

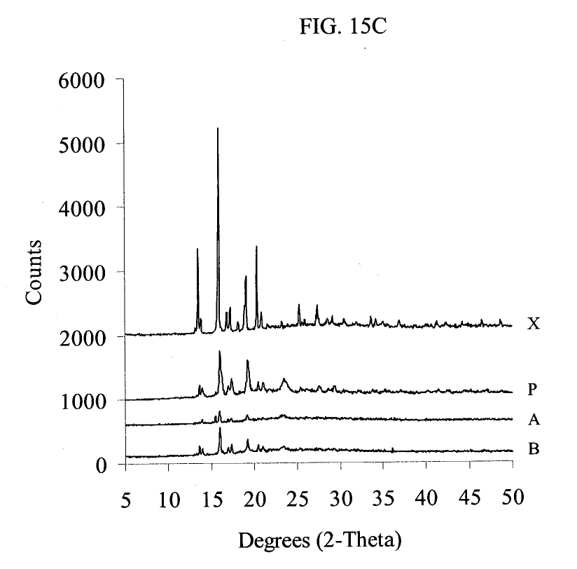

XRD patterns of the micronized SFL powders from emulsion are displayed in FIG. 15. Bulk danazol (FIG. 15a-X) is highly crystalline, producing intense peaks between 14 and 25 2θ degrees. The co-ground physical mixture (FIG. 15a-P) contained crystalline danazol peaks; however, the each micronized SFL powder from emulsion (FIGS. 15a-A, 15a-B, 15a-C, 15a-D and 15a-E) contained amorphous danazol. Crystalline danazol peaks could not be detected between 14 and 25 2θ degrees for these micronized SFL powders.

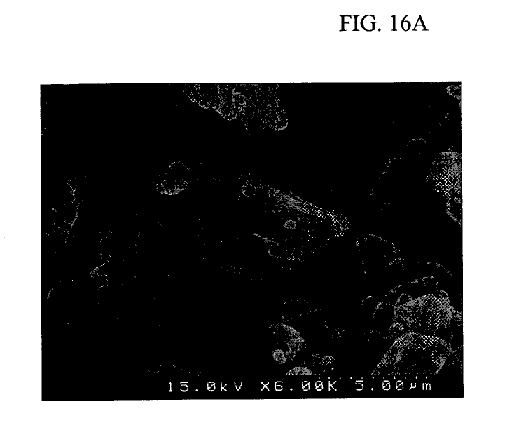

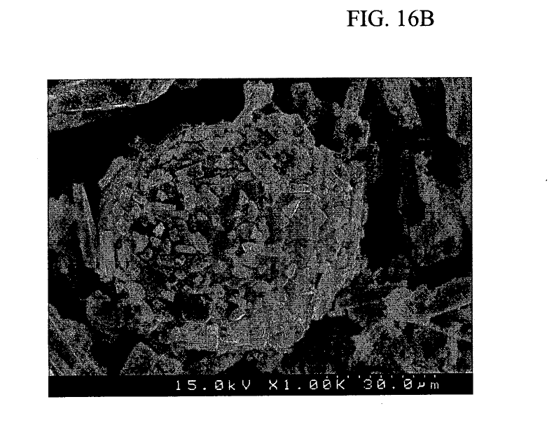

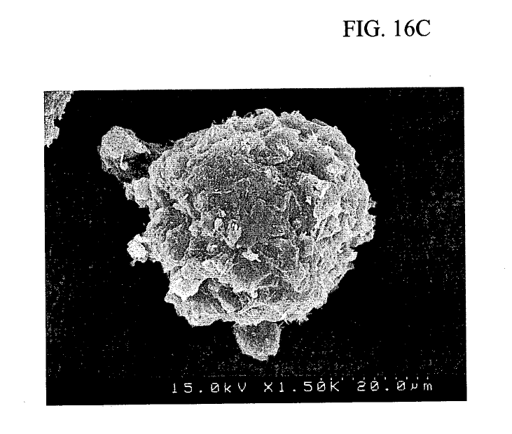

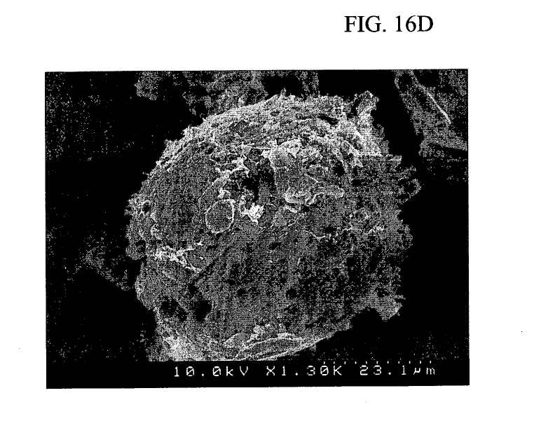

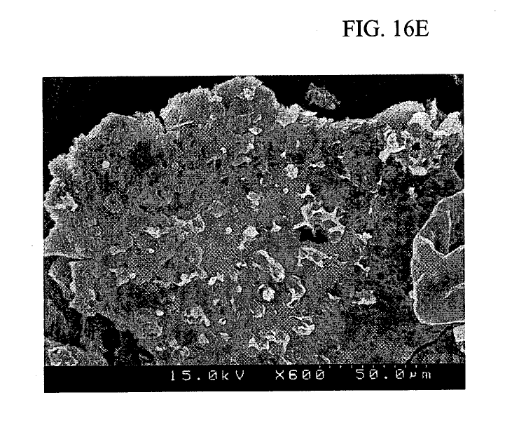

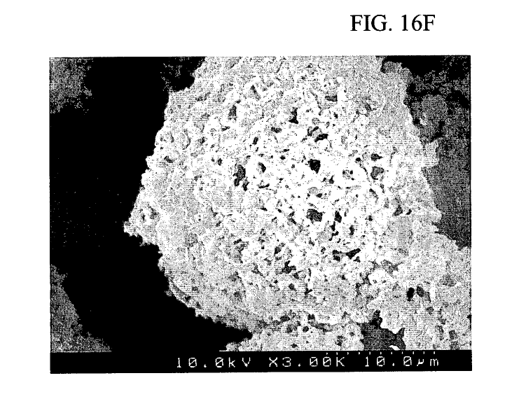

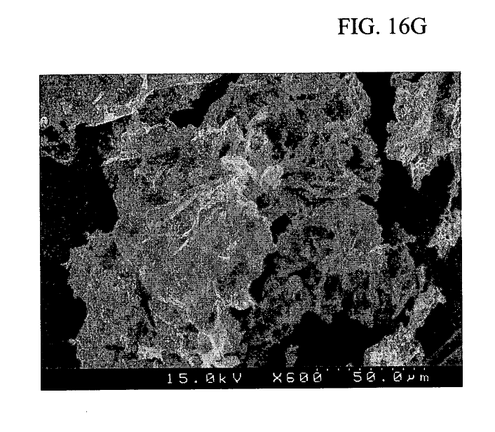

SEM micrographs of the various powders are shown in FIG. 16. As shown in FIG. 16a, bulk micronized danazol was composed of smooth, crystalline plates with fractured edges that ranged in length from 1 to 15 μm. The ingredients of the co-ground physical mixture (FIG. 16b) could be identified by comparison with pure components (SEM micrographs not shown). The spherical particle is poloxamer; the smooth acicular particles are PVA; the irregular particles with rough surfaces are PVP and the smooth, crystalline plates with fractured edges are danazol particles that were adsorbed to the surface of the poloxamer particle. An SEM of a representative micronized SFL A microparticulate aggregate is shown in FIG. 16c. The SFL aggregate was spherical with a rough surface, as was also the case for the SFL D microparticulate aggregate, shown in FIG. 16d

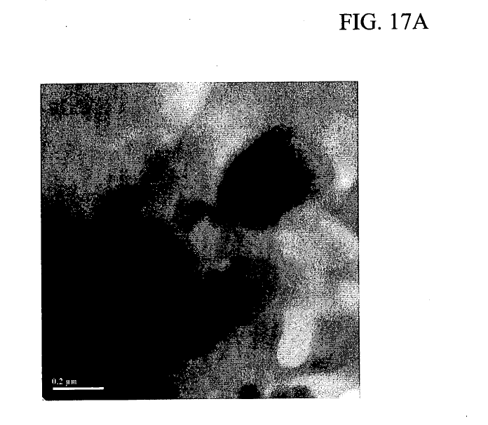

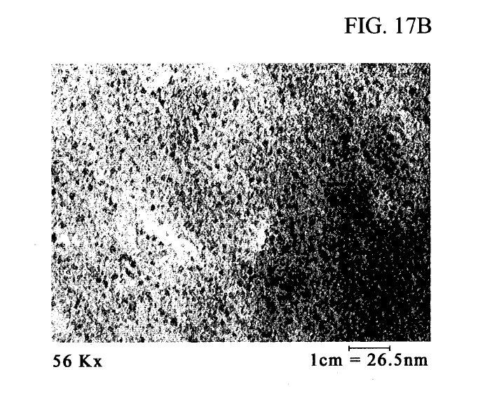

A TEM micrograph of the micronized SFL D powder is shown in FIG. 17a. The dark, electron-dense regions were determined by EDS spectra analysis to be danazol, and the lighter regions of lower electron density were determined to be the excipient matrix. Embedded within the excipient aggregate matrix of the dry micronized SFL D powder were small particle domains enriched in danazol that were approximately 200 nm in diameter. Towards the center of the SFL aggregate matrix, dense danazol-rich domains were observed as shown by the electron-dense center of the SFL microparticulate aggregate. These results indicated highly homogeneous mixing of the API and excipients.

Specific surface areas of the micronized SFL formulations are listed in Table 14. Whereas bulk danazol and the co-ground physical mixture had specific surface areas of 0.52 and 1.92 m²/g, respectively, the micronized SFL powders from emulsion had significantly higher surface areas (p<0.05). The specific surface areas of the micronized SFL A and SFL B powders were 12.71 and 41.73 m²/g, respectively, while that of the micronized SFL C powder was 83.06 m²/g. The surface areas of the micronized SFL D and SFL E powders were 20.18 and 24.98 m²/g, respectively.

Contact angle measurements of the various powders are listed in Table 14. The contact angle between SLS/Tris media and bulk micronized danazol was 64.3°. For the co-ground physical mixture, the contact angle was 41.8°, which was lower than that produced by bulk danazol (p<0.05). Representative samples of micronized SFL powders from emulsion had reduced contact angles with SLS/Tris media (p<0.05) compared to bulk danazol. Contact angles between the micronized SFL powders and SLS/Tris media ranged from 20.6° (micronized SFL E powder) to 48.0° (micronized SFL C powder), and the magnitude was influenced by the composition.

The dissolution profiles of the various powders investigated are shown in FIG. 5a. Approximately 53% of bulk danazol was dissolved within 5 minutes, and about 88% of bulk danazol was dissolved by 60 minutes. The co-ground physical mixture dissolved more slowly and to a lesser extent than bulk danazol (p<0.05). By 60 minutes, only 39% danazol had dissolved from the co-ground physical mixture. In contrast, greater than 90% danazol was dissolved from all micronized SFL powders by 5 minutes. The representative sample of micronized SFL powder from emulsion dissolved much more rapidly than bulk danazol or the co-ground physical mixture (p<0.05).

Investigation of slowly Frozen Agglomerates from Emulsion

The PSD measurements for the slowly frozen agglomerates from emulsion are listed in Table 14. The slowly frozen B agglomerate had an M50 of 20.12 μm, and the slowly frozen C agglomerate had an M50 of 9.50 μm. The M50 of the slowly frozen B agglomerate was higher than that of the C agglomerate (p<0.05).

Slowly frozen A and B agglomerates contained crystalline danazol, as indicated by the characteristic danazol peaks between 14 and 25 2θ degrees in the XRD patterns shown in FIGS. 2b-A and 2b-B, respectively. The slowly frozen C agglomerate contained amorphous danazol, as shown in FIG. 2b-C. The SEM micrograph of the slowly frozen A agglomerate is shown in FIG. 3e. SEM analysis of the slowly frozen formulations revealed large (>200 μm) agglomerates with smooth, continuous surfaces and fractured edges.

The specific surface areas and contact angles of the slowly frozen agglomerates are listed in Table 2. The slowly frozen B and C agglomerates had surface areas of 3.21 and 0.35 m²/g, respectively. The contact angles between the slowly frozen B and C agglomerates and SLS/Tris media were 23.6 and 47.8°, respectively, but the slowly frozen aggregates dissolved slowly and incompletely in the dissolution media within the 60-minute dissolution test period. The slowly frozen agglomerates dissolved more slowly and to a lesser extent than bulk danazol, but they dissolved faster and to a greater extent than the co-ground physical mixture (p<0.05). By 60 minutes, danazol contained in the slowly frozen A and B agglomerates were 72 and 83% dissolved in the dissolution media, while danazol contained in the slowly frozen C agglomerate was only 58% dissolved.

Investigation of Micronized SFL Powders and Slowly Frozen Agglomerates from Solution

The PSD measurement for the micronized SFL powder from solution is listed in Table 14. The M50 of the powder was 6.52 μm. As indicated in Table 14, the slowly frozen control produced large agglomerates that were difficult to reduce in size. In FIG. 2c, the micronized SFL powder from solution produced an XRD pattern (FIG. 15c-A) that indicated the presence of amorphous danazol. The slowly frozen control from solution produced an XRD pattern (FIG. 15c-B) revealed the presence of crystalline danazol as indicated by the characteristic peaks present between 14 and 25 2θ.

TABLE 14
Particle size distribution, specific surface area and contact angle measurements
of the dry micronized SFL powders and control formulations.

| | | | | | Specific | Average |
|---|---|---|---|---|---|---|
| | | | | | Surface Area | Contact Angle |
| Formulation | M 10(μm) | M 50(μm) | M 90(μm) | Span Index¹ | (m²/g) | (S.D.) |
| Bulk Danazol | 7.28 | 23.10 | 51.78 | 1.93 | 0.52 | 64.3 (1.1)² |
| SFL Control from Solution | 2.55 | 6.52 | 16.28 | 2.10 | 8.90 | 48.9 (2.8) |
| SFL A | 2.60 | 7.44 | 22.13 | 2.63 | 12.71 | 38.5 (2.1) |
| Slowly Frozen A | N/D³ | N/D | N/D | N/D | N/D | N/D |
| SFLB | 3.44 | 16.75 | 70.15 | 3.98 | 41.73 | 36.9 (1.0) |
| Slowly Frozen B | 4.31 | 20.12 | 60.56 | 2.80 | 3.21 | 23.6 (9.1) |
| SFLC | 2.98 | 6.07 | 12.42 | 1.55 | 83.06 | 48.0 (1.4) |
| Slowly Frozen C | 3.89 | 9.50 | 24.21 | 2.14 | 0.35 | 47.8 (2.1) |
| SFLD | 5.03 | 12.57 | 30.25 | 2.00 | 20.18 | 35.6 (2.1) |
| SFL E | 3.96 | 9.35 | 22.24 | 1.96 | 24.98 | 20.6 (11.9) |

¹Span index is (M 90 - M 10)/M 50 (18).
²It was determined that bulk danazol and danazol SFL processed in the absence of excipients gave similar contact angles and dissolution profiles.
³The slowly frozen A control was a solidified aggregate that could not be broken into small pieces sufficient for analysis

SEM analysis of the micronized SFL powder from solution (FIG. 16f) revealed a porous microparticulate aggregate comprised of small particle domains. SEM analysis of the slowly frozen agglomerate from solution (FIG. 16g) revealed a large (>200 μm) agglomerate with a smooth, continuous surface and fractured edges. TEM analysis of the micronized SFL powder from solution revealed small particle danazol domains that were approximately 20 nm in diameter. The electron-dense danazol domains were homogeneously dispersed throughout the porous excipient aggregate.

The specific surface area of the micronized SFL powder from solution is listed in Table 14. The micronized SFL powder from solution had a surface area of 8.90 m²/g, and the slowly frozen agglomerate had a surface area of 3.14 m²/g. The difference in contact angles for these two cases was relatively minor, as listed in Table 14.

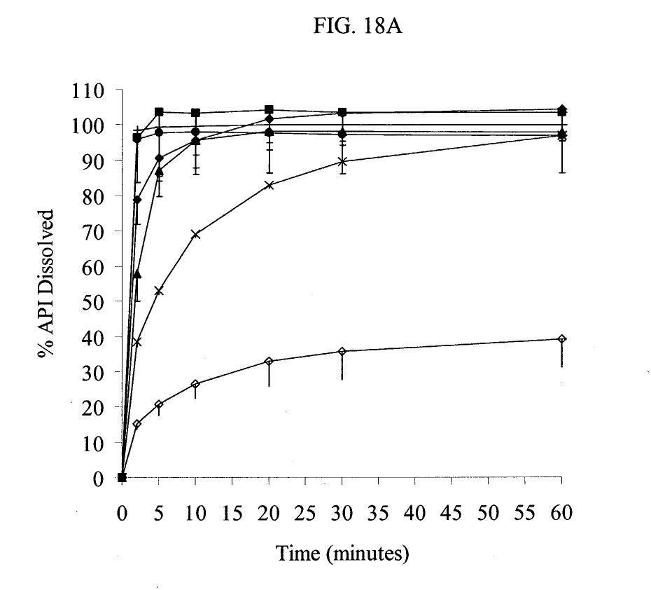

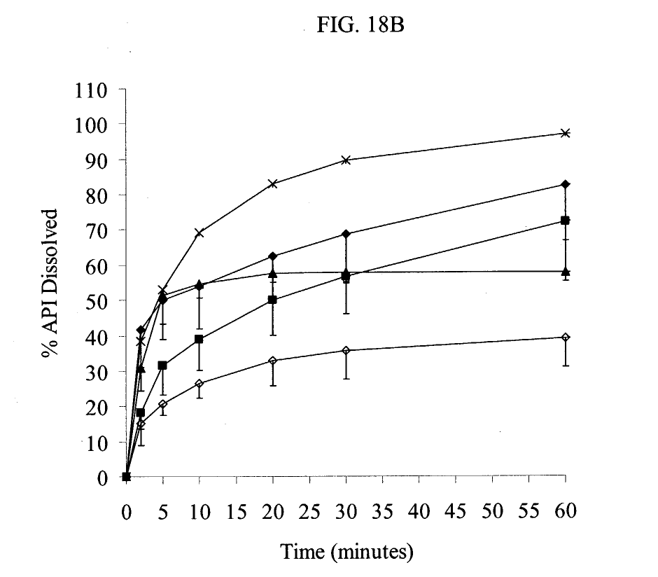

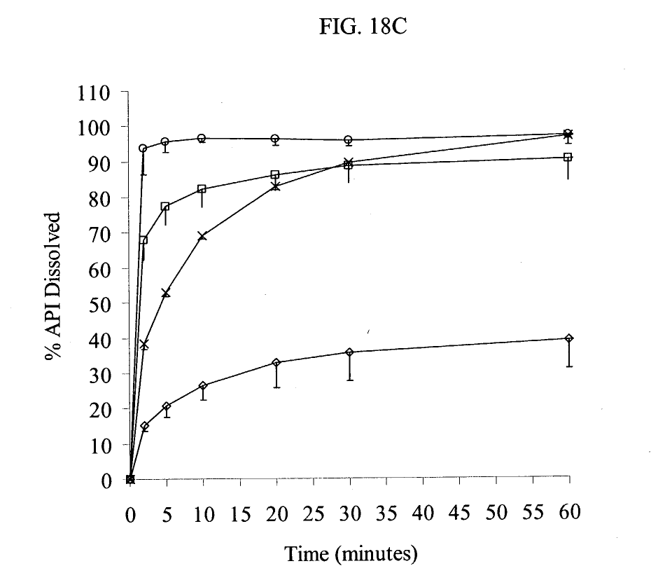

In FIG. 18c, the micronized SFL powder from solution displayed nearly complete dissolution (>90%) within 5 minutes, whereas the slowly frozen agglomerate did not reach 90% dissolution until 60 minutes. The micronized SFL powder from solution dissolved more rapidly than the slowly frozen agglomerate from solution (p<0.05). Both micronized SFL powder and slowly frozen agglomerate from solution dissolved more rapidly than bulk danazol or the co-ground physical mixture (p<0.05).

The micronized SFL powders produced by this process are characterized by high surface areas and contain amorphous API embedded in hydrophilic excipients that promote rapid and complete dissolution of the hydrophobic API. SFL processing offers the advantage of producing flowable micronized powders containing amorphous API in a single step. A micronized SFL powder with a 20:1:1:1 API-to-excipient ratio corresponded to an extremely large danazol potency of 87%.

From the XRD studies, it was demonstrated that SFL processing of emulsions produced micronized powders containing amorphous danazol. In addition, amorphous danazol was present in the micronized SFL D and SFL E powders, where danazol-to-excipient ratios were 20:1:1:1 and 50:1:1:1, respectively. Particle aggregation and crystallization were inhibited with unusually small excipient concentrations. SEM and TEM studies demonstrated that micronized porous aggregates consisting of small drug particle domains were produced by SFL processing of emulsions. Although SEM studies demonstrated that danazol, PVA, poloxamer and PVP were homogeneously blended and were not distinguishable from each other, TEM allowed the visualization of the distribution of the danazol small particle domains throughout core of the porous aggregate structure. From the electron-dense danazol domains shown in FIG. 17a, it can be seen that mass fraction of danazol was extremely high throughout the excipient matrix of the micronized SFL D formulation (10:1:1:1).

Near the edges of the porous aggregates, it became feasible to use high magnifications to view the individual danazol domains. Small particle danazol-rich domains measuring approximately 200 nm in size were clearly visible in the porous SFL microparticulate aggregate from emulsion. The domain sizes of the SEL powders produced from either emulsions or solution were sufficiently small such that wetting and dissolution were fast in both cases. This result is remarkable. Again, particle growth and crystallization were inhibited. The micronized SFL powders from emulsion that were highly loaded with danazol wetted and dissolved just as readily and completely as the micronized SFL powder from solution regardless of the differences in the particle domain size of danazol distributed throughout the porous aggregate matrices.

Large surface areas from 12.71 m²/g (SFL A) up to 83.06 m²/g (SFL C) were obtained with the micronized SFL powders, which were significantly higher than those of the slowly frozen agglomerates. The small domains of the freezing droplets in the spray led to faster freezing and greater preservation of high surface area of the precipitated solids. The surface areas of the micronized SFL powders increased as the concentrations of danazol and excipients in the emulsions increased.

The micronized SFL powders from emulsion wetted and dissolved rapidly compared to the slowly frozen agglomerates and control formulations (p<0.05). Even when higher API-to-excipient ratios were investigated, the micronized SFL D and SFL E powders dissolved greater than 90% of the danazol within 5 minutes in the dissolution media. Thus, it was demonstrated that micronized SFL powders with higher API-to-excipient ratios would wet and dissolve as readily as those micronized SFL powders with 10 times lower (20:1:1:1 vs. 2:1:1:1) API-to-excipient ratios when the SFL process was used to generate the powders. Regardless of the ratios investigated, the micronized SFL powders wetted and dissolved significantly more rapidly and completely compared to bulk danazol or the co-ground physical mixture (p<0.05).

The slowly frozen agglomerates from emulsion had significantly higher M50 particle sizes compared to the micronized SFL powders from emulsion (p<0.05). Because the agglomerates had to be mechanically broken up when dried, the primary particle sizes were larger than those sizes obtained from the SFL micronization process. Micronized powders were produced in a single step with SFL processing; so additional mechanical micronization was not necessary when micronized SFL powders were dried.

Slowly freezing the emulsions produced agglomerates that contained crystalline danazol, as demonstrated in the XRD studies. The EA-based slowly frozen agglomerates (A and B) were crystalline, but the DCM-based slowly frozen agglomerate (C) was amorphous.

From SEM analysis, it was shown that the slowly frozen agglomerates had different surface morphologies than the micronized SFL powders. Because the slowly frozen controls were composed of large agglomerates, the observed specific surface areas were significantly lower than those of the micronized SFL powders (p<0.05). The slowly frozen agglomerates wetted just as readily, but dissolved slowly and incompletely in aqueous media compared to the micronized SFL powders (p<0.05).

What is claimed is:

1. A protein or peptide particle composition having:
particles with an average size of about 5.0 nm to about 100 μm;
an average particle surface area greater than about 5.0 m²/g; and
less than about 10% monomer loss; and
an average particle alpha helix loss of less than about 20%.

2. The protein or peptide composition of claim 1, wherein the average particle surface area is greater than about 10 m²/g.

3. The protein or peptide composition of claim 1, wherein the average particle surface area is greater than about 15 m²/g.

4. The protein or peptide composition of claim 1, wherein the average percent monomer loss is less than about 5%.

5. The protein or peptide composition of claim 1, wherein the average percent monomer loss is less than about 3%.

6. The protein or peptide composition of claim 1, wherein the average percent monomer loss is less than about 0.8%.

7. The protein or peptide particle composition of claim 1, wherein the average particle alpha helix loss is less that about 15%.

8. The protein or peptide particle composition of claim 1, wherein the average particle alpha helix loss is less that about 10%.

9. The protein or peptide composition of claim 2, wherein the protein or peptide is selected from the group consisting of insulin, erythropoietin, growth hormone, follicle-stimulation hormone, leutinizing hormone, or a lysozomal storage enzyme.

10. A protein or peptide particle composition having:
particles with an average size of about 5.0 nm to about 100 μm;
an average particle surface area greater than about 5.0 m²/g; and
less than about 1% monomer loss.

11. A peptide or protein composition produced by spray freezing, wherein said particle composition has:
an average particle size of about 5.0 nm to about 100 μm;
an average particle surface area greater than about 5.0 m²/g; and
less than about 10% monomer loss; and
an average particle alpha helix loss of less than about 20%.

12. The protein or peptide composition of claim 11, wherein the average particle surface area is greater than about 10 m²/g.

13. The protein or peptide composition of claim 11, wherein the average particle surface area is greater than about 15 m²/g.

14. The protein or peptide composition of claim 11, wherein the average percent monomer loss is less than about 5%.

15. The protein or peptide composition of claim 11, wherein the average percent monomer loss is less than about 3%.

16. The protein or peptide composition of claim 11, wherein the average percent monomer loss is less than about 0.8%.

17. The protein or peptide particle composition of claim 11, wherein the average particle alpha helix loss is less that about 10%.

18. The protein or peptide particle composition of claim 11, wherein the average particle alpha helix loss is less that about 5%.

19. The protein or peptide composition of claim 11, wherein the protein or peptide is selected from the group consisting of insulin, erythropoietin, growth hormone, follicle-stimulation hormone, leutinizing hormone, or a lysozomal storage enzyme.

20. A peptide or protein composition produced by a process comprising:
mixing a protein or peptide with a solution agent;
spraying the protein or peptide-solution agent mixture into a cryogenic media at or below the surface of the cryogenic media so that the spray generates a protein or peptide particle composition;
the protein or peptide composition having:
an average particle size of about 5.0 nm to about 100 μm;
an average particle surface area greater than about 5.0 m²/g; and
less than about 10% monomer loss; and
an average particle alpha helix loss of less than about 20%.

21. The protein or peptide composition of claim 11, wherein the average particle surface area is greater than about 10 m²/g.

22. The protein or peptide composition of claim 11, wherein the average particle surface area is greater than about 15 m²/g.

23. The protein or peptide composition of claim 11, wherein the average percent monomer loss is less than about 5%.

24. The protein or peptide composition of claim 11, wherein the average percent monomer loss is less than about 3%.

25. The protein or peptide composition of claim 11, wherein the average percent monomer loss is less than about 0.8%.

26. The protein or peptide particle composition of claim 11, wherein the average particle alpha helix loss is less that about 10%.

27. The protein or peptide particle composition of claim 11, wherein the average particle alpha helix loss is less that about 5%.

28. The protein or peptide composition of claim 20, wherein the protein or peptide is selected from the group consisting of insulin, erythropoietin, growth hormone, follicle-stimulation hormone, leutinizing hormone, or a lysozomal storage enzyme.

29. A spray freezing process for producing particles comprising:
preparing a first emulsion comprising an encapsulation agent in a first solvent and an active agent in a first dispersed phase;
preparing a second emulsion comprising the first emulsion dispersed in a second solvent; and
spray freezing the second emulsion into a cryogenic liquid to produce the particles.

30. The spray-freezing process of claim 31, wherein the second emulsion is sprayed into the cryogenic liquid through an insulated nozzle at or below the level of the cryogenic liquid.

31. The spray freezing process of claim 31, wherein the second emulsion further comprises one or more excipients.

32. The spray freezing process of claim 31, further comprising the step of drying the particles.

33. A particle composition produced by the process of claim 31.

34. A method for spray freezing comprising:
mixing an active agent with an encapsulating agent to create a seed solution;
spraying the feed solution through an insulating nozzle located at or below the level of a cryogenic liquid;
generating frozen particles wherein the active agent is encapsulated by the encapsulating agent.

35. The method of claim 34 wherein the encapsulating agent is a polymer, co-polymer or polymer blend.
